SEQUENCE LISTING (8 sequences)

SEQ ID NO: 1 - DNA, 21 bp, synthetic construct
  source 1..21: mol_type = other DNA
aattctccgaacgtgtcacgt

SEQ ID NO: 2 - DNA, 21 bp, synthetic construct
  source 1..21: mol_type = other DNA
aacgtctatgtcaaatcgaca

SEQ ID NO: 3 - DNA, 21 bp, synthetic construct
  source 1..21: mol_type = other DNA
cagcctgtttgcagtggtcga

SEQ ID NO: 4 - DNA, 21 bp, synthetic construct
  source 1..21: mol_type = other DNA
ggaatctcattcgatgcatac

SEQ ID NO: 5 - DNA, 21 bp, synthetic construct
  source 1..21: mol_type = other DNA
ccaagctacttgagaatattt

SEQ ID NO: 6 - DNA, 21 bp, synthetic construct
  source 1..21: mol_type = other DNA
tctcgctcttgatagcaaatt

SEQ ID NO: 7 - DNA, 32 bp, synthetic construct
  source 1..32: mol_type = other DNA
agatcggaagagcacacgtctgaactccagtc

SEQ ID NO: 8 - DNA, 33 bp, synthetic construct
  source 1..33: mol_type = other DNA
agatcggaagagcgtcgtgtagggaaagagtgt

(19) United States
(12) Patent Application Publication
(10) Pub. No.: US 2026/0337275 A1
(43) Pub. Date: Oct. 29, 2026

(54) BLOCKING ITGB8 IN NEURODEGENERATIVE DISEASE

(71) Applicants: The Brigham and Women's Hospital, Inc., Boston, MA (US); The Regents of The University of California, Oakland, CA (US)

(72) Inventors: Amha Atakilit, San Francisco, CA (US); Oleg Butovsky, Needham, MA (US); Xiaoming Zhang, Brookline, MA (US); Neta Rosenzweig, Brookline, MA (US); Zhuoran Yin, Newton, MA (US); Dean Sheppard, San Francisco, CA (US); Thomas D. Arnold, San Francisco, CA (US)

(21) Appl. No.: 19/116,096

(22) PCT Filed: Sep. 25, 2023
(86) PCT No.: PCT/US2023/075059

§ 371 Date: Mar. 27, 2025

Related U.S. Application Data
(60) Provisional application No. 63/411,585, filed on Sep. 29, 2022.

(51) Int. Cl. C07K 16/28; A61K 39/00; A61P 25/28

(52) U.S. Cl. CPC C07K 16/2839; A61P 25/28; A61K 2039/505; C07K 2317/24; C07K 2317/76

(57) ABSTRACT

Provided herein are methods and compositions that block Integrin Subunit beta 8 (ITGB8, also known as integrin αvβ8) to treat neurodegenerative diseases associated with microglial impairment including Alzheimer's disease (AD) and amyotrophic lateral sclerosis (ALS).

### CLAIM OF PRIORITY

This application claims the benefit of U.S. Provisional Application Ser. No. 63/411,585, filed on Sep. 29, 2022. The entire contents of the foregoing are incorporated herein by reference.

### FEDERALLY SPONSORED RESEARCH OR DEVELOPMENT

This invention was made with Government support under Grant Nos AG051812, AG054672, AG075509, AG076982, AG080992, EY027921, NS088137, NS104609, and NS101673, awarded by the National Institutes of Health. The Government has certain rights in the invention.

### TECHNICAL FIELD

Provided herein are methods and compositions that block Integrin Subunit beta 8 (ITGB8, also known as integrin αvβ8) to treat neurodegenerative diseases associated with microglial impairment including Alzheimer's disease (AD) and amyotrophic lateral sclerosis (ALS).

### BACKGROUND

Microglia play an essential role in supporting normal brain functions¹, but in disease may contribute to neurodegeneration². Recently, genome-wide association meta-analysis studies (GWAS)^{3, 4} and interactome investigation⁵ identified microglia gene expression pattern to be strongly correlated with late onset Alzheimer's disease (AD). We identified that neurodegenerative microglia (MGnD)⁶, also referred to as disease associated microglia (DAM)⁷, are regulated by the reciprocal suppression of transforming growth factor b (TGFβ) and induction of apolipoprotein E (APOE) signaling in different neurodegenerative models including AD⁶. Human APOE has three major variants: e2, e3, and e4. APOE e4 is the major genetic risk factor for late-onset AD and was previously shown to accelerate AD progression in humans as well as in mouse models^{8, 9, 10, 11, 12, 13}. In the central nervous system (CNS), APOE is expressed in astrocytes, reactive microglia, oligodendrocytes, endothelial cells, and in the epithelial cells of the choroid plexus^{14, 15}. However, the role of APOE4 in the regulation of microglial phenotype and functions in vivo is unknown. APOE is a multifunctional protein in the CNS and within the periphery, where it is primarily expressed in liver and to a lesser extent in immune cells^{16, 17}. Although the deletion of APOE4 in hepatocytes did not affect amyloid deposition in APP/PS1 mice¹⁸, liver-specific expression of APOE4 on an Apoe knockout (KO) background enhanced AD pathology and impaired cognition in amyloid precursor protein (APP) transgenic mice¹⁹. Moreover, deletion of APOE4 in astrocytes markedly reduced neurodegeneration in a mouse model of tauopathy²⁰. Several studies investigated the molecular signature of microglia in mice globally expressing APOE4^{12, 13}, however, the cell-autonomous role of APOE4 expressed by microglia in the regulation of AD pathology has not been addressed.

### SUMMARY

APOE e4 is the strongest genetic risk factor for late-onset Alzheimer's disease (AD). The contribution of microglial APOE4 to AD pathogenesis is unknown, although APOE has the most enriched gene expression in neurodegenerative microglia (MGnD). Here we show, in mice and in humans, a negative role of microglial APOE4 in the induction of MGnD response to neurodegeneration. Deletion of microglial APOE4 restores MGnD phenotype, associated with neuroprotection in P301S tau transgenic mice and decreases pathology in APP/PS1 mice. Mechanistically, APOE4-mediated induction of ITGB8-TGFb signaling impairs MGnD response via upregulation of microglial homeostatic checkpoints, including INPP5D in mice. Microglial deletion of Inpp5d restores MGnD-astrocyte crosstalk and facilitates plaque clearance in APP/PS1 mice. We identified the microglial APOE4-ITGB8-TGFb pathway as a negative regulator of microglial response to AD pathology; as shown herein, restoring MGnD phenotype via blocking ITGB8-TGFb signaling can be used as a therapeutic intervention for neurodegenerative diseases including AD and ALS.

Thus provided herein are methods of treating a subject who has a neurodegenerative condition associated with microglial impairment, the method comprising administering a therapeutically effective amount of an inhibitor of Integrin Subunit beta 8 (ITGB8). Also provided herein are inhibitors of ITGB8 for use in a method of treating a subject who has a neurodegenerative condition associated with microglial impairment.

In some embodiments, the inhibitor of ITGB8 is an antibody that binds to ITGB8. In some embodiments, the antibody that binds to ITGB8 is ADWA11, ADWA16, C6D4, 37E11, HuC6D4F12, CL7290 or a humanized version thereof. In some embodiments, the humanized version is ADWA11-2.1, ADWA11-2.2, ADWA11-2.3, ADWA11-2.4, ADWA16-1, ADWA16-2, ADWA16-3, ADWA16-3.2, ADWA16-4, ADWA16hugraft, Ab1, Ab2, or Ab3.

In some embodiments, the inhibitor of ITGB8 is an inhibitory oligonucleotide targeting human ITGB8 that decreases ITGB8 expression. In some embodiments, the oligonucleotide is 15 to 21 nucleotides in length. In some embodiments, at least one nucleotide of the oligonucleotide is a nucleotide analogue. In some embodiments, the oligonucleotide is a gapmer or a mixmer.

In some embodiments, the neurodegenerative condition is Alzheimer's disease (AD) or amyotrophic lateral sclerosis (ALS).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-M APOE4 impairs microglial response to acute neurodegeneration. A, DEGs Heatmap of APOE4-KI vs. APOE3-KI microglia at 4 months of age with DEGs identified using DESeq2 analysis with LRT (n=11-14 mice/group, P<0.05). B, Spi1 normalized counts. C, Schematics of apoptotic neurons injection to the cortex and hippocampus of 8-months-old APOE3-KI and APOE4-KI mice; sorting strategy 16 hrs post-injection of phagocytic and non-phagocytic microglia for labelled apoptotic neurons; Created with Biorender.com. D, Gating strategy of CD11b⁺/Fcrls⁺ microglia from the injection site of apoptotic neurons (AN) in APOE3-KI and APOE4-KI mice. E, Bar plot showing percentage of CD11b⁺/Fcrls⁺ cells (n=7-9 mice/group). F, Principal component analysis (PCA) of each group. G, Heatmap of phagocytic and non-phagocytic microglia from APOE3-KI and APOE4-KI mice. DEGs were identified using DESeq2 analysis with LRT (n=4-6 mice/group, P<0.05). H, Gene ontology analysis of DEGs for phagocytosis, autophagosome maturation, IFNg signaling, and antigen presentation (P<0.05). I, Confocal microscopy images of Iba1, Lamp1 and AN at the injection sites. J, Quantification of Lamp1 immunoreactivity per Iba1+ cell (n=6-7 mice/group). K, Schematics of tamoxifen administration at 1.5 months of age, and the injection of AN to cortex and hippocampus of 8-months-old APOE3-KI, APOE4-KI, APOE3-cKO and APOE4-cKO mice. L, Percentage of CD11b⁺/Fcrls⁺ microglia in the injection site in APOE4-KI and APOE4-cKO mice (n=4 mice/group). M, Heatmap of non-phagocytic and phagocytic microglia isolated from APOE3-KI, APOE3-cKO, APOE4-KI, and APOE4-cKO injected with AN. DEGs were identified using DESeq2 analysis with LRT (n=3-6 mice/group, P<0.05). Two-tailed Student's t-test. Data were presented as mean #s.e.m.

FIGS. 2A-MAPOE4 impairs microglial response to neurodegeneration via PU.1. A, Schematics of Tat-Cre or PBS injection to the brains of Spi1^{fl/fl} followed by the injection of AN; Created with Biorender.com. B, Volcano plot showing DEGs in phagocytic microglia isolated from Spilfl/fl mice treated with Tat-Cre or PBS. DEGs were identified using DESeq2 analysis with LRT (n=5 mice/group, P<0.05). C, Scatter plot comparing the DEGs of microglia from Spi1-cKO and APOE4-cKO mice. DEGs were identified using DESeq2 analysis with LRT (P<0.05, Log2FC>0.25 or <−0.25) D, Representative images of brain sections from Tmem119^{WT/WT}: Spi1^{fl/WT}: APP/PS1 and Tmem119^{CreERT2/WT}: Spi1^{fl/WT}: APP/PS1 mice stained for HJ3.4B. E, Quantification of HJ3.4B⁺ area/ROI (n=3-6 mice/group). F, Confocal images of Clec7a, HJ3.4B and Iba1 in Tmem119WT/WT: Spi1fl/WT: APP/PS1 and Tmem119^{CreERT2/WT}: Spi1^{fl/WT}: APP/PS1 mice. G, Quantification of Clec7a⁺ and Iba1+ area per plaque (n=3-6 mice/group). H, Heatmap of microglia isolated from PU.1 inhibitor and control injected APP/PS1:APOE4-KI mice. DEGs were identified using DESeq2 analysis with LRT (n=4-5 mice/group, P<0.05). I, Confocal images of Iba1 and Clec7a in PU.1 inhibitor and control injected APP/PS1:APOE4-KI mice. J, Quantification of Clec7a immunoreactivity and Iba1⁺ area per ROI (n=4-5 mice/group). K, Normalized counts of Serpina3n in astrocytes isolated from PU.1 inhibitor and control injected APP/PS1:APOE4-KI mice (n=4-5 mice/group). L, Confocal images of Gfap and Serpina3n in PU.1 inhibitor and control injected APP/PS1:APOE4-KI mice. M, Quantification of Serpina3n+/Gfap+ area per ROI (n=4-5 mice/group). Two-tailed Student's t-test. Data were presented as mean±s.e.m.

FIGS. 3A-H Deletion of microglial APOE4 restores MGnD response to chronic neurodegeneration and promotes neuroprotection. A, Schematics of tamoxifen administration at 1.5 months of age and analysis of P301S mice at 9 months of age; Created with Biorender.com. B, qPCR validation of human APOE expression in sorted microglia (n=6-9 mice/group). C, DEGs of aggregated samples for WT and Tau (P301S) with APOE variants. DEGs were identified using DESeq2 analysis with LRT (n=3-11 mice/group, P<0.01). D, Confocal images of Clec7a, phospho-tau (AT-100) and Iba1. Arrowheads indicate Clec7a⁺ microglia associated with phospho-tau in the cortex in P301S:APOE3-KI, P301S:APOE3-cKO, P301S:APOE4-KI, and P301S:APOE4-cKO mice. E, Quantification of positive area of Clec7a and AT-100 in the cortex (n=7-16 ROIs/group). F, Volcano plots of DEGs of P301S:APOE4-cKO vs. P301S:APOE4-KI mice. DEGs were identified using DESeq2 analysis with LRT (n=3-8 mice/group, P<0.05). G, Representative images of Cresyl-violet staining of P301S mice carrying different APOE variants. Dashed squares indicate area of interest. H, Quantification of cortical neurons in WT and P301S mice carrying different APOE variants (n=7-10 mice/group). One-Way ANOVA. Data presented as mean±s.e.m.

FIGS. 4A-E Targeting microglial APOE4 restricts Ab pathology in APP/PS1 mice. A, Schematics of tamoxifen administration at 1.5 months of age and analysis of APP/PS1 mice at 4 months of age; Created with Biorender.com. B, qPCR validation of human APOE expression in sorted microglia (n=3-12 mice/group). C, Heatmap showing top 100 DEGs in microglia isolated from APP/PS1:APOE4-KI vs. APP/PS1:APOE4-cKO mice. DEGs were identified using DESeq2 analysis with LRT (n=5-9 mice/group, P<0.01). D, Quantification of cortical HJ3.4B⁺ plaque number/ROI (n=7-14 mice/group). E, Quantification of Lamp1 area in cortex (n=7-14 mice/group). One-way ANOVA. Data were presented as mean #s.e.m.

FIGS. 5A-M. Targeting microglial APOE4 promotes astrocyte activation and their recruitment towards plaque in APP/PS1 mice. a, UMAP plot of scRNAseq analysis of astrocytes showing clusters 3 and 5. Violin plots of key activation genes: Gfap, Vim, Fabp7, and Cd9 representing clusters 3 and 5. b, Volcano plot of cluster 3 comparing APP/PS1:APOE4-cKO vs. APP/PS1:APOE4-KI mice. DEGs were identified using FindMarkers Seurat function (P<0.05). c, Donut charts showing percentage of Gfap⁺Apoe^{low} and Gfap Apoe^{high} astrocyte clusters in APP/PS1:APOE4-KI vs. APP/PS1:APOE4-cKO mice. d, Top-upregulated canonical pathways in astrocytes from cluster 3 identified using IPA. DEGs for pathway enrichment analysis were determined using FindMarkers Seurat function with P<0.05 and pathways selected with P<0.05. e, Confocal images of Gfap, Iba1, and human APOE in the cortex of APP/PS1:APOE3-KI, APP/PS1:APOE3-cKO, APP/PS1:APOE4-KI and APP/PS1:APOE4-cKO mice at 4-months of age. Yellow arrows indicate human APOE immunoreactivity or its loss in Gfap+ and Iba1 cells. f, Quantification of Gfap+ area (n=39-61 plaques/group). g, Quantification of Apoe immunoreactivity in Gfap+ cells associated with plaques (n=39-61 plaques/group). h, Confocal images of Serpina3n, Gfap, and HJ3.4B. i, Quantification of Sepina3n⁺ immunoreactivity in Gfap+ cells (n=30-44 plaques/group). j, Schematics of experimental design of adoptive transfer of phagocytic microglia showing the isolation of MGnD from APOE3-KI, APOE4-KI and APOE4-cKO mice, and their injection to 2 months-old WT recipient mice, followed by the isolation of astrocytes from recipient mice 16 h later; Created with Biorender.com. k, Volcano plot showing DEGs of astrocytes isolated from WT recipient mice injected with MGnD microglia sorted from APOE4-KI compared with APOE3-KI mice. DEGs were identified using DESeq2 analysis with LRT (n=3 mice/group, P<0.05). I, Volcano plot showing DEGs of astrocytes isolated from WT recipient mice injected with MGnD cells sorted from APOE4-cKO mice compared with APOE4-KI mice. DEGs were identified using DESeq2 analysis with LRT (n=3 mice/group, P<0.05). m, IPA analysis of top-affected upstream regulators in WT astrocytes isolated from recipient mice following the injection of APOE4-cKO MGnD cells vs. APOE4-KI MGnD cells. DEGs used for upstream analysis were identified using DESeq2 analysis with LRT (n=3 mice/group, P<0.05). Upstream regulators with P<0.05 were selected. One-way ANOVA. Data presented as mean±s.e.m.

FIGS. 6A-L. Impaired induction of MGnD signature and astrocytes activation in APOE e4 AD carriers. Volcano plot of bulk-RNAseq analysis of total brain tissue isolated from male (a) and female (b) showing selected DEGs induced (red dots) and suppressed (blue dots) in AD APOE e3/4 carriers compared to AD APOE e3/3 carriers. DEGs were identified using DESeq2 analysis with LRT (male n=5-7 donors, female n=6-7 donors, P<0.05). c, Top-100 DEGs from female AD:APOE e3/4 carriers compared to AD:APOE e3/3 carriers. DEGs were identified using DESeq2 analysis with LRT (P<0.05, n=6-7 donors/group). d, Normalized counts of key affected genes. e, Top-affected KEGG pathways in female AD:APOE e3/3 carriers compared with AD:APOE e3/4 carriers. DEGs used for pathway analysis were identified with DESeq2 analysis and LRT. f, Volcano plot of microglial DEGs in AD:APOE e3/4 carriers compared with APOE e3/3 carriers analyzed from dataset by Zhou et al.⁴⁹. DEGs were identified using FindMarkers Seurat function (n=6 AD:APOE e3/3 carriers, n=4 AD:APOE e3/4 carriers, P<0.05). g, Confocal images of brain sections from APOE e3/3 and e3/4 AD females stained for pSmad3, IBA1, and HJ3.4B. h, Quantification of pSMAD3 immunoreactivity in IBA1+ cells (n=27-33 cells/group). i, Confocal microscopy images of GFAP in AD brains carrying APOE e3/3 and e3/4. j, Quantification of GFAP immunoreactivity per plaque (n=43-52 plaques/group). k, Donut plots representing analysis from dataset by Zhou et al.⁴⁹, showing percentage of GFAPHiSERPINA3″ and GFAPHiSERPINA3-astrocyte clusters in AD:APOE e3/4 and APOE e3/3 carriers (Average expression cutoff was GFAP>4, SERPINA3>0). l, Volcano plot of astrocytic DEGs of AD:APOE e3/4 carriers compared with APOE e3/3 carriers analyzed from dataset by Zhou et al.⁴⁹. DEGs were identified using FindMarkers Seurat function (n=6 AD:APOE e3/3 carriers, n=4 AD:APOE e3/4 carriers, P<0.05). White arrowheads indicate magnified ROIs for ig. Two-tailed Student's t-test. Data were shown as mean±s.e.m.

FIGS. 7A-P. Blocking ITGB8-TGFb signaling enhances MGnD response and reduces AD pathology in APP/PS1 mice. a, Representative images of Tmem119 immunoreactivity in sagittal brain sections of Itgb8-cKO mice and control littermates. Illustration indicates affected area (cortex) in blue. White arrowheads indicate magnified ROIs. b, Confocal microscopy images of Clec7a, Gfap, and Apoe in Itgb8-cKO and control mice. Quantification of (c) Clec7a* and (d) Gfap⁺ immunoreactivity in the cortex (n=12 ROIs/group). e, Heatmap of microglia isolated from Itgb8-KO and control mice. DEGs were identified using DESeq2 analysis with LRT (n=4-5 mice/group, P<0.05). f, Top KEGG Pathways in Itgb8-KO microglia compared with control microglia. DEGs for pathway analysis were identified using DESeq2 analysis with LRT and P<0.05 and pathways selected with P<0.05. g, Confocal images of pSmad3, Apoe, and Iba1 in Itgb8-cKO and control mice. White arrowheads indicate magnified ROIs. h, Quantification of pSmad3 immunoreactivity in Iba1+ cells (n=45-47 cells/group). i, Quantification of Apoe immunoreactivity in Iba1+ cells (n=45-47 cells/group). j, Scatter plot comparing the DEGs of microglia from Itgb8-cKO and Tgfbr2-cKO mice, described Lund et al.⁷⁵. DEGs for were identified using DESeq2 analysis with LRT (For Lund et al., n=3 mice/group; For Itgb8-cKO, n=4/5 mice/group; P<0.05, Log2FC>0.25 or <−0.25). k, Schematics of the administration of anti-ITGB8 neutralizing antibody or IgG isotype control to the brains of APP/PS1 mice and analysis 3 days later. l, Heatmap of microglia isolated from APP/PS1 mice treated with anti-ITGB8 neutralizing antibody and IgG isotype control, and top GO pathways affected. DEGs for were identified using DESeq2 analysis with LRT (n=5 mice/group, P<0.05). m, Confocal images of HJ3.4B⁺ plaques at the injection site 14 days after the treatment of APP/PS1 mice with anti-ITGB8 neutralizing antibody and IgG isotype control. n, Quantification of HJ3.4B⁺ plaques at the injection sites (n=8-9 mice/group). o, Confocal images of Gfap, Clec7a, and HJ3.4B⁺ plaques at the injection site 14 days after the treatment of APP/PS1:APOE4 KI mice with anti-ITGB8 neutralizing antibody and IgG isotype control. p, Quantification of HJ3.4B⁺ plaques area, Gfap+ area, and Clec7a+ area per ROI at the injection sites (n=11 ROIs from 6-7 mice/group). Two-tailed Student's t-test. Data were shown as mean±s.e.m.

FIGS. 8A-E. Impaired induction of MGnD signature and astrocyte activation in APOE e4 AD brains. a, Confocal images of GFAP and HJ3.4B immunoreactivity and detection of ITGB8 gene expression using RNAscope in AD:APOE e3/4 males compared to AD:APOE e3/3 males. Quantification of ITGB8 fluorescence in ROI (b) and in GFAP⁺ astrocytes (c) in AD:APOEe 3/4 males compared to AD:APOE e3/3 males (n=7-8 donors/group). d, Confocal images of GFAP and HJ3.4B immunoreactivity and detection of Itgb8 mRNA expression using RNAscope in APP/PS1:APOE4-cKO mice compared to APP/PS1:APOE4-KI mice. e, Quantification of ITGB8 fluorescence in GFAP⁺ astrocytes (n=10-18 ROIs from 3-5 mice per group). Two-tailed unpaired Student's t-test. Data were shown as mean±s.e.m.

FIGS. 9A-B. Blocking Itgb8 signaling enhances MGnD response and reduces AD pathology in APP/PS1 mice. A, Quantification of percentage of Ab-42 phagocytic microglia in WT and Itgb8-cKO mice (n=5-10 mice/group). B, MHC II, Iba1, and HJ3.4B in APP/PS1 mice were injected with anti-ITGB8 neutralizing antibody and IgG isotype control. The graph shows quantification of MHC II⁺ immunoreactivity at the injection site (n=4 mice/group). Two-tailed Student's t-test. Data were presented as mean±s.e.m.

FIGS. 10A-B. Microglial deletion of Smad2/3 induces MGnD phenotype. a, Expression of key homeostatic and MGnD genes in microglia from Itgb8-cKO (n=4-5 mice/group) relative to non-transgenic control mice. B, Expression levels of key AD-risk factor genes (Inpp5d, Havcr2 and Bin1) in microglia from Itgb8-cKO in microglia from Itgb8-cKO (n=4-5 mice/group) relative to non-transgenic control mice. Two-tailed Student's t-test. Data were shown as mean #s.e.m.

FIGS. 11A-D. Targeting ITGB8-TGFb signaling by ADWA-11 mitigates cognitive decline in 5×FAD mice. A, Schematics of anti-ITGB8 treatment and behavior tests (n=15). B, T-maze test evaluation. C, Latency evaluation in WT and 5×FAD mice during the five days trials in Water-maze. One-way analysis from the area under the curve. D, Time quantification (%) in the target quadrant on probe day at water-maze. One-way analysis. Data were shown as mean±s.e.m. *P<0.05, **P<0.01.

### DETAILED DESCRIPTION

Several mechanisms have been proposed by which APOE4 increases the risk of developing AD, most involving models harboring global APOE4 expression or astrocyte specific deletion^{12, 13, 20, 58}. Here we show a cell-autonomous negative regulation of microglial APOE4 in the acquisition of MGnD phenotype in response to neurodegeneration. The induction of MGnD response was restored in mice with tau and amyloid pathology in which microglial APOE4 was deleted, resulting in improved neuronal survival, reduction in plaque pathology, and increased microglial and astrocytic association with Aβ plaques. Deletion of microglial APOE3 reduced Clec7a expression to levels comparable to APOE4-KI in P301S mice. We recently showed impaired MGnD responses in APOE4-KI glaucoma mice, similarly to Apoe^{-/-} mice²². Thus, microglial expression of APOE3 is critical for the induction of MGnD response to neurodegeneration and that its deletion accelerated neuronal loss, whereas deletion of microglial APOE4 favored neuroprotection in tau mice. In addition to the classical role of APOE in cholesterol transport, APOE4 was shown to acquire a novel function in the nucleus, where it translocated and directly bound DNA to affect the transcription of genes, associated with AD^{59, 60} Furthermore, microglial APOE4 showed increased nuclear localization in AD brains⁵⁹. These results suggest that the nuclear localization of APOE4 may directly repress the transcription of MGnD genes, a function absent in APOE3 microglia, which is alleviated following the deletion of APOE4. Thus, APOE may have a dual role in controlling MGnD response based on its subcellular localization, which may be altered in APOE4 expressing microglia, accounting for the different impact on neurodegeneration detected in tau mice following the deletion of microglial APOE3 compared to APOE4. Consistent with these findings, we found that the transcription factor PU.1 was upregulated in APOE4-KI microglia, and its microglial deletion resulted in a robust induction of MGnD genes. Moreover, using microglia-specific expression of human APOE variants, Liu et al., demonstrated that microglial APOE3 elicits a transcriptomic signature of activated immune response, whereas APOE4 impairs activation of inflammatory responses to amyloid pathology. This APOE4 microglial phenotype was associated with reduced expression of genes involved in antigen presentation and interferon responses and increased Aβ plaque deposition. In AD brains carrying the APOE e4 allele, we found a sex-dependent impairment of MGnD signature with a pronounced reduction in astrocyte activation in females. Several AD risk genes including CD33, BINI, ABCA7, CRI, INPP5D and HAVCR2^{4, 61} were enriched in the brains of APOE e4 AD females, associated with downregulation of key MGnD genes^{6, 21, 22}. These results support previous reports demonstrating that higher PU.1 expression in humans is likely to result in an earlier AD onset^{29, 62}. In female AD brains of APOE e4 allele, PU.1 expression was associated with increased SMAD3 expression levels, which would reinforce microglial homeostatic signature as a downstream molecule of TGFb signaling^{21, 28, 50}. Moreover, immunoreactivity phosphorylated SMAD3 (pSMAD3) as a marker of TGFb signaling was increased in microglia in the female AD brains of APOE e4 allele, indicative of the promotion of microglial homeostatic signature. TGFb enriched milieu in the AD brain of APOE e4 carriers suppresses astrocyte activation, consistent with previous reports showing astrocyte suppression in response to TGFb ligand⁶³ and deletion of Tgfbr2⁴⁴ or Smad2/3 activates astrocytes. We also found that microglial deletion of APOE4 in APP/PS1 mice promotes astrocyte activation and encapsulation of Ab plaques. Moreover, reactive astrocytes were shown to play a beneficial role in limiting AD pathology, and their depletion in AD mice resulted in increased plaque load, synaptic dysfunction, and memory loss⁶⁴. These findings in mice and in humans suggest that APOE4 predisposes to AD pathology in part via the induction of TGFb-dependent microglia homeostatic regulators²¹ that impair microglia-astrocyte crosstalk in response to neurodegeneration. In line with these findings, Liu et al., demonstrated that APOE4 reduces activated microglia signatures responses to AD pathology in human brains and human induced pluripotent stem cell-derived microglia of APOE e4 allele.

Although TGFb signaling has been suggested to play both beneficial and detrimental roles in AD⁷², its overproduction in astrocytes promoted cerebrovascular fibrosis and amyloidosis⁷³. Moreover, TGFb1 was implicated in vascular dementia to promote abnormal vascular remodeling and was proposed as a therapeutic target for AD⁷⁴. We detected upregulation of ITGB8, which plays a critical role in the activation of latent TGFb 1⁵¹, in AD male of APOE e4 carriers. In female AD brains carrying the APOE e4 allele, we identified induction of SMAD3 and INPP5D signaling associated with downregulation of MGnD genes. Mechanistically, the genetic deletion of Itgb8 or Inpp5d was sufficient to restore MGnD response and astrocytic activation, coupled with reduced plaque load in AD mice. Furthermore, pharmacological blocking of ITGB8-TGFb signaling enhanced MGnD response associated with increased plaque clearance in AD mice. These data support the beneficial role of MGnD-microglia in restricting AD pathology and identify ITGB8-TGFb axis as a therapeutic intervention for AD.

### Methods of Treatment

Provided herein are methods of reducing microglial impaired response to neurodegeneration in a subject and for treating neurodegenerative diseases associated with microglial impairment. The present methods can be used for any mammalian subject, e.g., human subjects. Thus provided herein are methods for treating a subject in need thereof, e.g., a subject who has microglial impairment and/or a neurodegenerative disease associated with microglial impairment, that include administering to the subject an effective amount of an ITGB8 inhibitor.

In some embodiments, the neurodegenerative disease associated with microglial impairment is Alzheimer's disease (AD) or amyotrophic lateral sclerosis (ALS). In some embodiments, the subject has Alzheimer's disease or another tauopathy such as frontotemporal dementia, frontotemporal dementia with Parkinsonism, frontotemporal lobe dementia, multiple system tauopathy, multiple system tauopathy with presenile dementia, Wilhelmsen-Lynch disease, disinhibition-dementia-parkinsonism-amytrophy complex, Pick's disease, or Pick's disease-like dementia, corticobasal degeneration, frontal temporal dementia, Parkinson's disease, Huntington's disease, amyotrophic lateral sclerosis (ALS), or multiple sclerosis (MS), e.g., progressive MS. In some embodiments, the disease is, or is not, an eye related neurodegenerative disease such as glaucoma and age-related macular degeneration.

As used herein, a “therapeutically effective amount” is an amount sufficient for reducing signs or symptoms of a disease, reducing (slowing) progression of a disease, reducing severity of a disease, in a subject diagnosed with the disease. A “prophylactically effective amount” is an amount that reduces the incidence or risk of a sign or symptom of a disease in a subject at risk for the disease, or delays onset of sign or symptom of the disease in a subject who is at risk, e.g., who has a genetic mutation associated with a disease as described herein. A sign or symptom can include dementia, forgetfulness/memory loss. A subject as described herein can be a human, who has been diagnosed with a neurodegenerative disease as described herein, or as having a mutation associated with a neurodegenerative disease as described herein.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### ITGB8 Inhibitors

A number of ITGB8 inhibitors are known in the art, including antibodies and inhibitory oligonucleotides.

### Anti-ITGB8 Antibodies

Antibodies and antigen-binding fragments thereof that bind to human ITGB8 include CL7290 (described in WO2022268644), 37E1 antibodies described in U.S. Pat. No. 9,290,572; ADWA-2, ADWA-8, ADWA-10, ADWA-11, ADWA-13, ADWA-15, ADWA-16, ADWA-25, and ADWA-20 (described in US20160046717); C6D4 and other antibodies such as B13C4 15-8, B13C4 15-10, B13H3.2, B13C1231015, B15B11VH, B2B2 15-9, R11D12715.3, RSDLVH-1, RSDLVH-3, RSDLVH-16, both 29 and 44, A1-B4=F9, A5-C6, and D4-E6 (described in WO2018064478); HuC6D4F12 (described in US20210277125), and variants thereof, including humanized and chimeric versions thereof. In some embodiments, the antibody is a humanized ADWA11 antibody described in WO2020051333 (including ADWA11 VH01/VK01, ADWA11 VH02/VK01, ADWA11 VH03/VK01, ADWA11 VH03/VK02, ADWA11 VH05/VK01, ADWA11 VH05-2/VK01, ADWA11-2.1, ADWA11-2.2, ADWA11-2.3, and ADWA11-2.4) and humanized ADWA16 antibody (including ADWA16-1, ADWA16-2, ADWA16-3, ADWA16-3.2, ADWA16-4, ADWA16hugraft, Ab1, Ab2, or Ab3 described in WO2022164816). Additional antibodies may be known in the art or obtained using methods known in the art, including those described in the references above.

In some embodiments, the antibody or antigen-binding fragment thereof specifically binds human and blocks binding of TGFp peptide to αvβ8.

### Inhibitory Oligonucleotides Targeting ITGB8

As described above, the methods can include the administration of inhibitory oligonucleotides (“oligos”) targeting ITGB8 (i.e., ITGB8 mRNA or DNA) that reduce ITGB8 expression. Oligos useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), mixmers, gapmers, and other oligomeric compounds or oligonucleotide mimetics that hybridize to at least a portion of ITGB8 and modulate its function. In some embodiments, the oligos include antisense RNA, antisense DNA, chimeric antisense oligonucleotides, antisense oligonucleotides comprising modified linkages, interference RNA (RNAi), short interfering RNA (siRNA); or a short, hairpin RNA (shRNA); or combinations thereof. See also WO 2015/051239.

Sequences for human ITGB8 are known
in the art and include the following:

| Nucleic Acid NCBI RefSeq ID | Protein NCBI RefSeq ID |
|---|---|
| NM_002214.3 | NP_002205.1 |

An exemplary genomic sequence for human ITGB8 is at NC_000007.14, range 20329766 to 20415754.

In some embodiments, the oligos hybridize to at least 7, 8, 9, 10, 11, 12, 13, 14, 15, 16, 17, 18, 19, 20, 21, 22, 23, 24, 25, 26, 27, 28, 29, 30 or more consecutive nucleotides of the target sequence.

In some embodiments, the methods include introducing into the cell an oligo that specifically binds, or is complementary, to ITGB8. A nucleic acid that binds “specifically” binds primarily to the target, i.e., to ITGB8 RNA but not to other non-target RNAs. The specificity of the nucleic acid interaction thus refers to its function (e.g., inhibiting ITGB8) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. These methods can be used to treat a subject, e.g., a subject at risk for neurodegeneration following acute injury or with evidence of a chronic neurodegenerative disease, by administering to the subject a composition (e.g., as described herein) comprising an oligo that binds to ITGB8. Examples of ITGB8 target sequences are provided above. Exemplary sequences include those commercially available, e.g., shRNA from Santa Cruz Biotechnology, as well as siRNA as follows: AATTCTCCGAACGTGTCACGT, AACGTCTATGTCAAATCGACA, and CAGCCTGTTTGCAGTGGTCGA (S′-3′), or shRNA shB8-1, or shB8-4 as follows: GGAATCTCATTCGATGCATAC, CCAAGCTACTTGAGAATATTT, and TCTCGCTCTTGATAGCAAATT (S′-3′) (Malric et al., Mol Cancer Res. 2019 February; 17 (2): 384-397).

In some embodiments, the methods described herein include administering a composition, e.g., a sterile composition, comprising an oligo that is complementary to ITGB8 sequence as described herein. Oligos for use in practicing the methods described herein can be an antisense or small interfering RNA, including but not limited to an shRNA or siRNA. In some embodiments, the oligo is a modified nucleic acid polymer (e.g., a locked nucleic acid (LNA) molecule), a gapmer, or a mixmer.

Oligos have been employed as therapeutic moieties in the treatment of disease states in animals, including humans. Oligos can be useful therapeutic modalities that can be configured to be useful in treatment regimens for the treatment of cells, tissues and animals, especially humans.

For therapeutics, an animal, preferably a human, suspected of having or being at risk of neurodegeneration is treated by administering an oligo in accordance with this disclosure. For example, in one non-limiting embodiment, the methods comprise the step of administering to the animal in need of treatment a therapeutically effective amount of an oligo as described herein.

In some embodiments, the oligos are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such oligos; for example, an oligo 30 nucleotides in length may have a portion of 15 bases that is complementary to the targeted ITGB8 RNA. In some embodiments, the oligonucleotides are 15 nucleotides in length. In some embodiments, the antisense or oligonucleotide compounds described herein are 12 or 13 to 30 nucleotides in length. One having ordinary skill in the art will appreciate that this embodies oligos having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the oligo comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligos are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligos described herein may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligo comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ~N(CH₃)~O~CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

### Pharmaceutical Compositions and Methods of Administration

The methods described herein can include the administration of pharmaceutical compositions and formulations comprising ITGB8 inhibitors and/or oligonucleotides designed to target ITGB8.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The oligos can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions described herein include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., inhibitory nucleic acid sequences described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of inhibitory nucleic acid sequences described herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In some embodiments, these injectable oil-in-water emulsions comprise a paraffin oil, a sorbitan monooleate, an ethoxylated sorbitan monooleate and/or an ethoxylated sorbitan trioleate.

The pharmaceutical compounds can also be administered by intranasal, routes including insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111).

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or intrathecal administration. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an oligo can be made by lyophilizing a solution comprising a pharmaceutical described herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations described herein can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods described herein are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In some embodiments, the methods described herein can include co-administration with other drugs or pharmaceuticals. For example, the inhibitor of ITGB8 can be co-administered with drugs for treating or reducing risk of a disorder described herein.

### EXAMPLES

The invention is further described in the following examples, which do not limit the scope of the invention described in the claims.

### Materials and Methods

The following materials and methods were used in the Examples below.

### Human Brain Tissue and Neuropathology

Frozen brain tissue blocks from the superior parietal gyrus for RNAseq and chromatin immunoprecipitation DNA sequencing (ChIPseq) were obtained from the Netherlands Brain Bank (NBB). Donors gave informed consent to donate their brains to NBB. Post-mortem interval was limited to 10 hours to ensure tissue quality. Paraffin brain tissue sections were obtained from NBB and Massachusetts Alzheimer's Disease Resource Center (ADRC). The study was approved by the Ethics Committees of the Brigham Women's Hospital, University of California San Francisco, and compliant with the World Medical Association Declaration of Helsinki on Ethical principles for medical research involving human subjects. For tissue selection, neuropathological staging of the brains was performed by NBB and ADRC.

### Mice

B6.129P2 (Cg)-Cx3cr1^{tm2.1(cre/ERT2)Litt}/WganJ mice (Cx3cr1-CRE^{ERT2}, stock #021160)²⁶, C57BL/6-Tmem119^{em1(cre/ERT2)Gfng}/J (Tmem119-CRE^{ERT2}, stock #031820-)⁷⁶, B6.129S6-Inpp5d^{tm1Wgk}/J mice (Inpp5d^{flox}, stock #028255)⁷⁷, Smad2^{tm1.1Epb}/J mice (Smad2^{flox}, stock #022074)⁷⁸ and Smad3^{tm1Zuk} mice (Smad3^{flox}, MGI: 3822465)⁷⁹ were purchased from the Jackson Laboratory. Cx3cr1-CREERT2 mice were crossed with human APOE knock-in mice, APOE3^{fl/fl} and APOE4^{fl/fl} mice, respectively¹⁸. These transgenic mice were then triple crossed with APP/PS1³⁴ or P301S³² mice. B6.Cg-Tg (Thy1-APPSw, Thy1-PSEN1*L166P)21Jckr (APP/PS1)³⁴ mice were kindly provided by Dr. Mathias Jucker (University of Tubingen). B6;C3-Tg (Prnp-MAPT*P301S) PS19Vle/J (P301S, Jax stock #008169)³² mice have been backcrossed to C57BL/6 mice for more than ten generations. B6.129P2-Apoe^{tm2(APOE*3)Mae} N8 (MGI #4838571)⁸⁰ and B6.129P2-Apoe^{tm3(APOE*4)Mae} N8 (MGI #4838572)⁸¹ were obtained from Taconic. The Spi1^{fl/fl} mice⁸² were generously provided by Daniel Tenen and Junyan Zhang from Beth Israel Deaconess Medical Center. The Itgb8^{fl/fl} mice⁸³ were kindly provided by Dr. Thomas Arnold from University of California, San Francisco. The Itgb8-tdT mice were provided by Dr. Helen Paidassi⁸⁴ from Université Claude Bernard Lyon 1. Cx3cr1-CRE^{ERT2} mice were crossed with Inpp5d^{fl/fl} mice both on WT and APP/PS1 background. Cx3cr1-CRE (stock Tg(Cx3cr1-cre) MW126Gsat/Mmucd, MMRRC_036395-UCD) mice^{85, 86} were obtained from the Mouse Resource and Research Center (MMRRC) at University of California Davis, and were crossed with Smad2^{flox} and Smad3^{flox} mice. All the experimental procedures using animals were approved by the Institutional Animal Care and Use Committee at Brigham and Women's Hospital, Harvard Medical School.

### Tamoxifen Treatment

At six weeks of age, mice were intraperitoneally (ip) injected with 75 mg/kg of tamoxifen (Sigma Aldrich, T5648-5G) dissolved in corn oil per day for five consecutive days.

### Microglia Isolation

Mice were euthanized in a CO₂ chamber and transcardially perfused with ice-cold Hanks' Balanced Salt Solution (HBSS, Thermo Fisher, 14175103). The whole brain was removed from the skull and separated into sections with a sagittal brain matrix for further processing. The left hemisphere was used for sorting and the right hemisphere was used for immunohistochemistry. The left hemisphere was homogenized to form a single cell suspension, then resuspended and centrifuged in a 37%/70% Percoll Plus (GE Healthcare, 17-5445-02) gradient in HBSS at 800 g, 23 °C, for 25 min with an acceleration of 3 and a deceleration of 1. Mononuclear cells were taken from the interface layer. The cells were stained with rat APC-conjugated anti-mouse Fcrls (1:1000, clone 4G11, Butovsky Lab) 21, PE-Cy7-conjugated anti-mouse CD11b (1:300, eBioscience, 50-154-54), and PerCP/Cy5.5-conjugated anti-mouse Ly-6C (1:300, Biolegend, 128012). After staining, Ly-6C⁻CD11b⁺Fcrls⁺ cells were washed and sorted using BD FACSAria™ II (BD Bioscience). The analysis was done using FlowJo™ 10. For mice with stereotaxic injections of apoptotic neuron (AN), a 3×6 mm² coronal slice around the injection site was taken. Microglia were enriched using the same method mentioned above. The apoptotic neurons were labeled with Alexa Flour™ 405 dye. During sorting, phagocytic (AN-Alexa Fluor 405 Ly-6C-CD11b⁺Fcrls⁺) and non-phagocytic (AN-Alexa Fluor 405⁻Ly-6C⁻CD11b⁺Fcrls⁺) microglia were collected as previously described⁶.

### Primary Neuron Culture

Primary Neurons were obtained from mouse embryos at embryonic day 18 (E18). Cerebral hemispheres were isolated. The meninges were then removed from the hemispheres under a dissection microscope. The cerebral hemispheres were digested in a 10 U/mL papain solution for 15 min at 37 °C, and then triturated in a trituration solution of 19 mL Neurobasal media (Gibco, 21103-049), 133 μL BSA (Sigma, A-9576), 0.5 mL Pen/Strep/Glucose/Pyruvate, and 200 μl DNase (Sigma, D-5025) with fire-polished glass pipettes until a single cell suspension was created. The suspension was filtered through a 40-μm cell strainer, spun down at 400 g for 5 min, and resuspended in 1 mL HBSS. The cell number was counted using a trypan blue stain (Gibco™, 15-250-061) and a cellometer (Nexcelom). Cells were plated on a 6 well, poly-D-lysine coated plate with approximately 1 million cells per well in a minimum volume of 2 mL growth media made of Neurobasal, 1:50 B-27 supplement (Gibco 17504-044), 1:200 Pen/Strep (Gibco, 15140-122), 1:400 Glutamax (Gibco, 35050-061), and 1:50 fetal bovine serum (FBS, Gibco, 10-438-026). After 24 h of incubation at 37 °C, cell viability and contamination were checked under the microscope. Half of the growth media was replaced with fresh growth media containing 5 μM Ara-C(Sigma-Aldrich, C1768-100 MG) without FBS. After five days, half of the media was removed and replaced with new growth media without FBS.

### Induction of Apoptotic Neurons

Primary neurons (d7-d10) were typically cultured one w after the initiation of culture. Neurons were removed from the surface of the plate by multiple washing with PBS. The neurons were then incubated under UV light (302 nm) at an intensity of 6315 W for 20 min to induce apoptosis. After this step, the neurons were kept on ice. The cells were collected, spun down via centrifugation, and resuspended in 1 mL PBS. Next, they were stained with the labeling dye (Alexa Fluor™ 405 NHS Ester, Invitrogen, A3000) for 15 min at 37 °C, protected from light. Neurons were then washed, spun, and resuspended. The number of apoptotic cells was determined using a trypan blue stain and a cellometer. Neurons were resuspended at a density of 25,000 dead cells per μL PBS.

### RT-qPCR

Total RNA from mouse tissue was extracted using RNeasy Plus Micro Kit (Qiagen, 74034) according to the manufacturer's protocol. Total RNA from human tissue was extracted using mir Vana miRNA isolation kit (Invitrogen, AM1560) according to the manufacturer's protocol. For quantitative reverse transcription polymerase chain reaction (RT-qPCR), RNA was used after the reverse transcription reaction (high-capacity cDNA Reverse Transcription Kit; Applied Biosystems, 436884). Total mRNA amplifications were performed with commercially available FAM-labeled Taqman probe for human APOE (Thermo Fisher Scientific, Hs00171168_m1) and mouse Gapdh (Thermo Fisher Scientific, Mm9999915_g1). A real-time PCR reaction was performed using QuantStudio™ 7 (Applied Biosystems). All RT-qPCRs were performed in duplicate, and the level of mRNAs were presented as relative expression normalized to Gapdh as mean±SEM.

### Stereotaxic Injection

Mice were anesthetized via intraperitoneal injection of ketamine (100 mg/kg) and xylazine (10 mg/kg). To induce MGnD paradigm, 2 μl of the apoptotic neuron mixture was injected into the hippocampus (ML: +1.5 mm, AP:-2 mm, DV:-2 mm) and cortex (ML: +1.5 mm, AP:-2 mm, DV:-1 mm) bilaterally using a stereotactic injection apparatus. Animals were processed 16 hours after injection. To knockout PU.1 from microglia, 6 to 8-week-old PU.1^{flox/flox} mice were stereotaxically injected with TAT-Cre (MilliporeSigma, SCR508) into ventricles (ML: +1 mm, AP:-1 mm, DV: −2.5 mm; 2 μl per injection site, 10 μg/μl, 40 μg per brain). To induce MGnD paradigm, 5 days after the i.c.v. injection of TAT-Cre, Alexa Flour™ 405 dye labelled apoptotic neurons, premixed with TAT-Cre, were injected into the cortex and hippocampus as described above. Phagocytic and non-phagocytic microglia were sorted from the brain regions narrow down to the injection sites, followed by SmartSeq2 analysis. To study the role of PU.1, we sued PU.1 inhibitor (DB1876, MCE, HY-135797A) to inhibit PU.1. PU.1 inhibitor was dissolved in 10% DMSO (Invitrogen, D12345) to get the concentration as 2.08 mg/ml (4 mM) according to the manufacture's instruction. 10% DMSO was used as control. The PU.1 inhibitor or control was injected into the cortex and hippocampus as described above. After 7 days, microglia and astrocytes were isolated from the brain regions restricted to the injection sites, followed by IHC and Smart-seq2 analysis. To study the role of MGnD carrying different APOE alleles in the regulation of astrocytes response, the apoptotic neurons were injected into the cortex and hippocampus of APOE3-KI, APOE4-KI, and APOE4-cKO mice to induce MGnD paradigm as described above. After 16 hours, phagocytic microglia were sorted out of the brains, then injected into the hippocampus and hippocampus of WT mice hippocampus (ML: +1.5 mm, AP:-2 mm, DV: −1 and −2 mm; 2 μl per injection site), 3000 microglia per injection site. After 16 hours, astrocytes were isolated from the brain regions restricted to the injection sites, followed by Smart-seq2 analysis. To study the role of Itgb8, an anti-ITGB8 antibody (ADWA11) was used to block mouse Itgb8. Mouse IgG1 was used as isotype control (BioXCell, Catalog #BP0083). Both ADWA11 and IgG1 were diluted to 0.5 μg/μl in sterile PBS. The anti-ITGB8 antibody or isotype control was injected into the cortex and hippocampus (ML: +1.5 mm, AP:-2 mm, DV:-1 and-2 mm; 2 μl per injection site). After 14 days, microglia were isolated from the brain regions restricted to the injection sites, followed by IHC and Smart-seq2 analysis. Fluorescence-labeled amyloid-b peptide (1-42) (Anaspec, AS-60480-01) were reconstituted by NH₄OH, then diluted in sterile PBS to 1 μg/μL. The Ab was injected into the cortex and hippocampus (ML: ±1.5 mm, AP: −2 mm, DV: −1 and −2 mm; 2 μl per injection site). After 6 hours, phagocytic and non-phagocytic microglia were isolated from the brain regions restricted to the injection sites, followed by FACS analysis.

scRNA-Seq

Brain tissue isolated from mice following perfusion was performed as previously described⁸⁷. Briefly, tissue was finely minced and incubated in 10 mL of enzyme digestion solution consisting of 75 μL Papain suspension (Worthington, LS003126) diluted in enzyme stock solution (ESS) and equilibrated to 37 °C. 200 mL ESS medium consisted of 20 mL 10×EBSS (Sigma-Aldrich, E7510), 2.4 mL 30% D (+)-Glucose (Sigma-Aldrich, G8769), 5.2 mL 1M NaHCO₃(VWR, AAJ62495-AP), 200 μL 500 mM EDTA (Thermo Fisher Scientific, 15575020), and 168.2 mL ddH2O, filter-sterilized through a 0.22-μm filter. Samples were shaken at 80 rpm for 40 min at 37 °C. Enzymatic digestion was stopped with 1 mL of 10× hi ovomucoid inhibitor solution and 20 μL 0.4% DNase (Worthington, LS002007) diluted in 10 mL inhibitor stock solution (ISS). 10 mL 10× hi ovomucoid inhibitor stock solution contained 300 mg BSA (Sigma-Aldrich, A8806), 300 mg ovomucoid trypsin inhibitor (Worthington, LS003086) diluted in 10 mL 1×PBS and filter sterilized using at 0.22-μm filter. ISS medium contained 50 mL 10×EBSS (Sigma-Aldrich, E7510), 6 mL 30% D (+)-Glucose (Sigma-Aldrich, G8769), 13 mL 1M NaHCO₃(VWR, AAJ62495-AP) diluted in 170.4 mL ddH₂O and filter-sterilized through a 0.22-μm filter. Tissue was mechanically dissociated using a 5 mL serological pipette and filtered through at a 70-μm cell strainer (Thermo Fisher Scientific, 22363548) into a fresh 50 ml conical tube. Tissue was centrifuged at 500 g for 5 min and resuspended in 10 mL of 30% Percoll solution (2.7 mL Percoll Plus (GE Healthcare Biosciences, 17-5445-01), 1 mL 10×PBS, 6.3 mL ddH2O). The samples were centrifuged at 800 g, 23 °C, for 25 min with an acceleration of 4 and a deceleration of 3. Samples were loaded onto a 10× Genomics Chromium platform for GEM and cDNA generation carrying cell- and transcript-specific barcodes and sequencing libraries constructed using the Chromium Single Cell 3′ Library & Gel Bead Kit v3. Libraries were sequenced on the Illumina. NovaSeq S1 was used targeting a depth of 100,000 reads per cell. Gene counts were obtained by aligning reads to the mm10 genome (refdata-gex-GCRm38-2020-A) using CellRanger software (v.4.0.0) (10× Genomics). Single-cell clustering and differential expression analyses were carried out using Seurat (v.4.0.6) (satijalab.org/Seurat/index.html)⁸⁸ Cells with more than 200 sequencing reads and less than 20 percent mitochondrial transcripts were selected, and genes with more than two reads across all samples entered downstream analyses. Expression counts were normalized by the “LogNormalize” method and scaled for mitochondrial read count using linear regression as implemented in Seurat's “Regress Out” function. Variable genes were identified using the “vst” selection method. The data was then centered and scaled, analyzed by principal component analysis (PCA), and dimensionally reduced to the top 30 principal components. The cells were clustered on PCA space using gene expression data as implemented in FindNeighbors and FindClusters commands in Seurat-v4. The method returned 27 cell clusters which were then visualized on a UMAP created by the top 30 principal components. The differentially expressed genes in each cluster were output by FindAllMarkers. The clusters were identified based on cell-type-specific key signature genes. 26 clusters were confidently assigned with one cluster showing no confident cell-type signature. For identified astrocytic cells (clusters 3 & 5), the differentially expressed genes in cluster 3 and cluster 5 comparison were identified by using the FindMarkers Function. Donut plots for astrocytes were created using a cutoff of >2 for Gfap and Apoe>3, Vim>1, Serpina3n>0.9, Cd9>0.9. For identified microglial cells (clusters 1, 4, 9 & 26), re-clustering of the cells was performed to extract any lost variations from the original clustering of all cells. The data were centered and scaled, analyzed by principal component analysis (PCA), and dimensionally reduced to the top 26 principal components. The method returned 11 cell clusters which were then visualized on a UMAP created by the top 26 principal components. The differentially expressed genes in homeostatic and MGnD sample comparisons were identified by cell-type-specific key signature genes using the FindMarkers Function. Gene expression was visualized using FeaturePlot, DittoHeatmap, and VlnPlot functions from Seurat-v4. Processed data from Zhou et al.⁴⁹ and Olah et al.⁴⁸ was downloaded from the AD Knowledge Portal and processed using workflow described above with some adjustments. For Zhou et al.⁴⁹, cells with 5% mitochondrial content or more were removed. Cell types were annotated using the scType⁸⁹. For Olah et al. only cells with less than 10 percent mitochondrial transcripts were selected. (For detailed steps, see ‘Code availability’).

### Bulk RNA-Seq

Smart-Seq2 libraries were prepared by the Broad Technology Labs and sequenced by the Broad Genomics Platform. cDNA libraries were generated from sorted cells using the Smart-seq2 protocol⁹⁰. RNA sequencing was performed using Illumina NextSeq500 using a High Output v2 kit to generate 2×25 bp reads. Count files (fastq) were downloaded and aligned using Salmon (v1.7) to mm10 genome and checked for sequencing quality using Multiqc (v1.11). Potential technical outliers were removed for further analysis. All analysis was carried out using DESeq2 (v.1.34.00)⁹¹. Biological outliers were determined using PCA plotting and heatmap visualization and removed for final analysis. Low abundance genes below a mean count of 5 reads per sample were filtered out. Comparisons were run using LRT, and the cutoff for significant genes was either P<0.01 or P<0.05. Heatmaps were visualized using the pheatmap package (v.1.0.12), volcano plots were generated using the EnhancedVolcano package (v1.12.0), and violin plots were generated using the function geom_violin from the ggplot2 package (v3.3.5) (For detailed steps see ‘Code availability’).

### Astrocytes Sorting

Astrocytes were isolated following perfusion using enzymatic digestion as described in the isolation of mouse brain cells for scRNA-seq. Cells were stained for 30 minutes in the dark on ice. The following antibodies were used for negative selection: PE anti-CD45R/B220 (BD, 553089, 1:100), PE anti-Ter119 (Biolegend, 116207, 1:100), PE anti-Olig4 (R&D Systems, FAB1326P, 1:100), PE anti-CD105 (eBioscience, 12-1051-82, 1:100), PE anti-CD140a (eBioscience, 12-1401-81, 1:100), PE anti-Ly6G (Biolegend, 127608, 1:100), PE-Cy7 anti-CD11b (eBioscience, 50-154-54, 1:300), BV421™ anti-CD45 (Biolegend, 103133, 1:100) and Alexa Fluor® 700 anti-01 (R&D Systems, FAB1327N, 1:100). APC anti-ACSA2 (Miltenyi Biotec, 130-117-535, 1:100) was used for positive selection of sorted astrocytes.

### Cross-Dataset Comparison

Bulk RNAseq data from Lund et al.⁷⁵ were cross-compared with our Itgb8-KO data in WT mice. DEGs (P<0.05, Log₂ Fold Change (FC)> (0.25)) were selected from both studies and DEGs were plotted on a scatter plot. MGnD-related genes were determined by Kraseman et al. 20176. Linear regression was calculated using geom_smooth ( ) for MGnD genes and M0 genes (For detailed steps, see ‘Code availability’).

### Ingenuity Pathway Analysis

Pathway analysis was performed using gene ontology (GO) enrichment analysis (geneontology.org). Differentially expressed genes were used to detect the pathways associated with biological processes. Differentially expressed genes with corresponding fold changes and adjusted p values were applied to gene set enrichment analysis (GSEA, gsea-msigdb.org/gsea/index.jsp)^{92, 93} and Ingenuity pathway analysis (IPA, digitalinsights.qiagen.com/products-overview/discovery-insights-portfolio/analysis-and-visualization/qiagen-ipa/). In IPA, canonical pathways and biological functions were tested for generating biological networks as described previously²¹.

### Nuclei Isolation

The brain nuclei were isolated from frozen human brains following the methods previously developed⁹⁶ with mild modification. Briefly, about 100 mg brain tissue were dissected on dry ice and immediately homogenized in 10 ml 1% formaldehyde. The homogenate was fixed for exact 10 min with shaking and then quenched by adding Glycine with final concentration as 0.125 M. Then the fixed brain homogenate was washed twice with NF1 buffer⁹⁶ and lysed for 60 min on ice. After further dissociation using Dounce homogenizer and filtering with a 70 μm cell strainer, the nuclei were placed on top of sucrose cushion and centrifuged to remove the myelin. The nuclei pellet was then washed twice with FACS buffer (HBSS with 0.2% BSA) and finally was snap frozen for future use.

### Anti-H3K9Ac ChIP-Seq

The anti-H3K9ac ChIP was performed using iDeal ChIP-seq kit for Histones (diagenode, C01010059) following the manufacturer's instruction with mild modifications. The sonication program was 20 cycles (30 seconds “ON”, 30 seconds “OFF”) at a high-Apower setting. Two ml of anti-H3K9ac antibody (Millipore, 07-352) was added per IP reaction. The DNA libraries were prepared using NEBNext® Ultra™ II DNA Library Prep Kit for Illumina® (NEB, E7103S), following the manufacturer's instructions. The DNA libraries were analyzed using Qubit 4 Fluorometer (Invitrogen, Q33238) and 2100 Bioanalyzer DNA system (Agilent). The pooled libraries were sent to Genewiz, Inc. and sequenced 2×150 bp in Illumina HiSeq platform.

### ChIP-Seq Data Analysis

Raw fastq files were first checked for quality using Multiqc sequence analysis. Cutadapt (v.4.0) was used to cut adaptors (-a AGATCGGAAGAGCACACGTCTGAACTCCAGTC-A AGATCGGAAGAGCGTCGTGTAGGGAAAGAGTGT), and reads were aligned to the mouse genome (mm10) using bowtie2 (2.3.4.3)⁹⁷. Subsequently, SAM files were sorted and filtered using Sambamba (v.0.8.2)⁹⁸. We discarded unmapped and duplicate fragments. Sorted BAM files were indexed using samtools (v.1.15.1)⁹⁹. BAM files were then converted to BigWig files for visualization using deeptools (v.3.5.0) 100. Normalization for all BigWig files were carried out against effective genome size (2652783500 for mice H3K9ac ChIP-seq). Peak analysis was carried out with Macs2 (v.2.2.7)¹⁰¹ using stringent parameters (-f BAMPE—mfold 5 50-p 0.001). A master-peak file was created for extracting counts from BAM files. For mouse H3K9ac, microglia isolated from 3 mice were pooled for ChIP. The threshold of peak pile for both samples was set to >10 and the cutoff for fold change was set at >2 or <0.5 to determine significantly different peaks (For more details, see ‘Code availability’). Peaks were visualized using IGV (v.2.11.4), exported as .png, and edited in Adobe Illustrator.

### Lipidomics

Analyses of polar and non-polar lipids were conducted using a liquid chromatography/mass spectrometry (LC-MS) system comprised of a Shimadzu Nexera X2 U-HPLC (Shimadzu Corp.) coupled to an Exactive™ Plus orbitrap mass spectrometer (Thermo Fisher Scientific). 30K sorted microglia were prepared in 50 μl isopropanol. After centrifugation, 10 μL supernatant was injected directly onto a 100×2.1 mm, 1.7 μm ACQUITY BEH C8 column (Waters). The column was eluted isocratically with 80% mobile phase A (95:5:0.1 vol/vol/vol 10 mM ammonium acetate/methanol/formic acid) for 1 minute followed by a linear gradient to 80% mobile-phase B (99.9:0.1 vol/vol methanol/formic acid) over 2 minutes, a linear gradient to 100% mobile phase B over 7 minutes, then 3 minutes at 100% mobile-phase B. Mass spectrometry (MS) analyses were carried out using electrospray ionization in the positive ion mode using full scan analysis over 200-1100 m/z at 70,000 resolution and 3 Hz data acquisition rate. Other MS settings were: sheath gas 50, in source CID 5 eV, sweep gas 5, spray voltage 3 kV, capillary temperature 300 °C, S-lens RF 60, heater temperature 300 °C, microscans 1, automatic gain control target 1e6, and maximum ion time 100 ms. Raw data were processed using TraceFinder software (Thermo Fisher Scientific) for targeted peak integration and manual review of a subset of identified lipids and using Progenesis QI (Nonlinear Dynamics) for peak detection and integration of both lipids of known identity and unknowns. Lipid identities were determined based on a comparison to reference extracts and are denoted by the total number of carbons in the lipid acyl chain(s) and total number of double bonds in the lipid acyl chain(s). All analysis was carried out using R (v.4.1.1). A total of 199 metabolites were reduced to 170 by removing any metabolites with missing data in one or more of the samples. Data were normalized using the z-score method. The heatmap of the 170 metabolites was plotted using the pheatmap package (v.1.0.12).

### Immunohistochemistry

Mice were transcardially perfused with cold HBSS before tissue excision and fixation. Tissues that were not adequately perfused were not further analyzed to eliminate autofluorescence associated with blood contamination. Two different tissue preparation protocols (paraffin-embedded for human brain tissue or microtome free-floating sections) were applied, as previously described³³. Briefly, sections were blocked in PBS with 20% horse serum (Thermo Fisher Scientific, NC9909742) and 0.3% Triton X-100 (Sigma) for 1 hour at room temperature. Primary antibodies were incubated overnight at 4 °C in PBS containing 2% horse serum and 0.3% Triton X-100. The following primary antibodies were mouse anti-Aβ (1:300, BioLegend, 803001); chicken anti-GFAP (1:400, Abcam, 4674); mouse anti-Phospho-Tau (1:50, Thermo Fisher Scientific, AT-100, MN1060); goat anti-Iba1 (1:100, Abcam 5076); rabbit anti-Iba1 (1:200, Wako, 019-19741); mouse anti-HJ3.4B¹⁰² (1:600, Holtzman Lab); rat anti-LAMP1 (1:100, DSHB, 1D4B); rat anti-Dectin1 (1:100, Invivogen, Clone: R1-8g7); rabbit anti-APOE (1:400, Cell Signaling Technology, 13366S); rabbit anti-pSmad3 (1:100, Abcam, ab52903); guinea pig anti-Plin2 (1:200, Fitzgerald industries international, 20R-AP002); goat anti-Serpina3n (1:200, R&D systems, AF4709) and anti-MHC II (1:200, BioLegend, I-A/I-E, 107601). Secondary antibodies included: Cy2/Cy3/Cy5-conjugated donkey anti-mouse/goat/rabbit/rat/chicken/guinea pig antibodies (1:200; all from Jackson Immunoresearch). Sections were imaged on a Zeiss LSM710 confocal using 20× or 40× objective. Two negative controls were routinely used in immunostaining procedures, staining with isotype control antibody followed by the secondary antibody or staining with the secondary antibody alone. The tissues were applied to slides, mounted with ProLong™ Gold Antifade Mountant with DAPI (Thermo Scientific, P36931) or with VECTASHIELD® Antifade Mounting Medium (Vector Laboratories, H-1000), and sealed with coverslips. The mouse brains of Itgb8-tdT and Smad2/3-cKO line were harvested following transcardial perfusion with 20 mL cold PBS and 20 mL cold 4% formaldehyde. Tissue was fixed in 4% formaldehyde overnight at 4 °C, followed by overnight incubation in 30% sucrose. Samples were embedded (Tissue-Plus™ O.C.T. Compound, Fisher Scientific, 23-730-571) and cryosectioned at 20 μm. Sections were mounted onto glass, blocked with PBS containing 1-2% BSA, 5% donkey serum and 0.5% TritonX-100. Primary and secondary antibodies were diluted in PBS containing 1% BSA and 0.25%-0.5% TritonX-100. The primary antibodies were goat anti-Sox9 (1:300, R&D systems, AF3075); rabbit anti-Olig2 (1:300, Millipore, AF2418); mouse anti-NeuN (1:300, Millipore, MAB377); rabbit anti-GFAP (1:300, DAKO, Z0334); rat anti-GFAP (1:300, Invitrogen 13-0300); goat anti-Pdgfra (1:300, R&D Systems, AF1062); Goat anti-Iba1 (1:300, Novus, NB100-1028); Rat anti-Cd68 (1:300, Bio-Rad, MCA1957); Rabbit anti-Apoe (1:300, Abcam, ab183596). Secondary antibodies included: Cy2/Cy3/Cy5-conjugated donkey anti-mouse/goat/rabbit antibodies (1:200; Jackson Immunoresearch).

### Thioflavin-S Staining and Quantification

Free-floating brain sections with a section thickness of 30 μm were incubated in filtered 1% aqueous Thioflavin-S(Sigma T1892) for 8 minutes at room temperature. The sections were then sequentially washed in 80%, 95% ethanol and distilled water. Entire cortical images were taken with Leica Microsystems DMi8 Microscope using Tile scan. The positive area of Thioflavin-S was quantified using the automatic thresholding method in Fiji with “Otsu”. 7 animals were analyzed per group.

### Cresyl Violet Staining and Neuron Counting

Free-floating brain sections with a section thickness of 30 μm were stained with Cresyl violet (Sigma) to estimate neuronal survival. Staining was performed to visualize neurons. Sample slices were incubated in 100% ethanol for 6 min and defatted in xylene for 15 min, followed by another 10 min in 100% ethanol. After rinsing in distilled water, the slides were stained with 0.5% Cresyl violet acetate for 15 min and again rinsed in distilled water. The sections were then placed in differentiation buffer (0.2% acetic acid in 95% ethanol) for 2 min, dehydrated with ethanol and xylene, and mounted with Depex medium. Pyramidal neurons were counted in each brain from serial sections located 30 μm apart and analyzed using One-way ANOVA with Fisher's LSD post hoc test. The % neuronal survival is quantified relative to the number of the pyramidal neurons in the age-matched APOE3-KI mice.

### RNAscope

RNAscope® ISH technology was applied on human formalin-fixed, paraffin-embedded (FFPE) sections and mouse 4% PFA-fixed (fixed-frozen) sections using RNAscope® Multiplex Fluorescent Reagent kit (v2, ACD, 323100), ITGB8 human probe (Hs-ITGB8-XMfa, ACD, 515881), Itgb8 mouse probe (Mm-Itgb8, ACD, 407931), INPP5D probe (Hs-INPP5D, ACD, 465051), negative control probe DapB (ACD, 321831), and positive control probe (Hs-PPIB, ACD, 313901) according to the manufacturer instructions. The sections were further blocked with 5% BSA, 5% Normal Donkey serum, 0.3% Triton in PBS for 1 hour, followed by primary antibody overnight at 4 °C (anti-GFAP, 1:400, Abcam, 4674; anti-IBA1, 1:200, Wako, 019-19741; anti-HJ3.4B102, 1:600, Holtzman Lab). The secondary antibodies (AlexaFluor 488 Donkey anti-mouse, 1:300, AlexaFluor 546 Donkey anti-goat, 1:300, AlexaFluor 546 Donkey anti-rabbit, 1:300, AlexaFluor 647 Donkey anti-mouse, 1:300) were applied for 2 hours. After PBS wash, the sections were mounted with Fluoromount-G™ mounting medium (Thermo Fisher, 00-4958-02) with DAPI and sealed with coverslips.

### Image Visualization

Images were cropped, merged, and optimized using Fiji, Photoshop CS6 13.0 (Adobe) and were arranged using Adobe Illustrator CS5 15.1.

### Image Analysis

To assess the amount of MGnD and neuritic dystrophy, randomly selected 40× magnification images were taken close to the injection sites of APOE3-KI, APOE4-KI, and APOE4-cKO mice. The positive area of Lamp1 of APOE3-KI and APOE4-KI mice were quantified using the automatic thresholding method in Fiji with “Otsu”. The positive area of Lamp1 was further normalized to the positive area of each Iba1⁺ cell. 5-7 animals were analyzed per experimental group. To quantify the HJ3.4B⁺ plaque load in Tmem119^{CreERT2/WT}: Spi1^{fl/wt}. APP/PS1 and Tmem119^{WT/WT}: Spi1^{fl/wt}: APP/PS1 mice, 10× magnification images were taken from the cortex. The positive area of HJ3.4B was quantified using the automatic thresholding method “RenyiEntropy” in Fiji. The positive areas of Clec7a and Iba1 were quantified using the automatic thresholding method “Moments” in Fiji. 3-6 mice were analyzed per experimental group. To assess the amount of Clec7a, Iba1 and Serpina3N, 10× and 40× magnification images were taken from the cortex of PU.1 inhibitor or control injected APP/PS1:APOE4-KI mice. The integrated density of the Clec7a of was measured using Fiji. The automatic thresholding methods in Fiji were used to quantify the positive areas (“Moments” for Iba1, “Triangle/Triangle” for Serpina3N/Gfap). To assess the amount of Clec7a and phospho-Tau (AT-100), 40× magnification images were taken from the cortex of P301S:APOE3-KI, P301S:APOE3-cKO, P301S:APOE4-KI, and P301S:APOE4-cKO mice. The positive areas of Clec7a and AT-100 were quantified using the automatic thresholding method in Fiji with “Triangle”. 7-16 ROI from 4 animals were analyzed per experimental group. To assess the amount of APOE, 40× magnification images were taken from the cortex of P301S:APOE4-KI and P301S:APOE4-cKO mice. The positive area of APOE was quantified using the automatic thresholding method in Fiji with “Triangle”. Five mice per group were analyzed per experimental group. To measure the area of Clec7a, Gfap, Apoe, Serpina3n and Plin2 in the plaque area, 20× or 40× magnification images were taken from the cortex of APP/PS1:APOE3-KI, APP/PS1:APOE3-cKO, APP/PS1:APOE4-KI, and APP/PS1:APOE4-cKO mice. The images were exported to Fiji and individual plaques were selected and cropped for analysis. The positive area of Clec7a and Gfap per plaque were quantified using automatic thresholding methods in Fiji (“RenyiEntropy” for Clec7a, “Otsu” for Gfap). The positive area of Clec7a and Gfap was further normalized to plaque size in each image. 55-73 plaques for Clec7a and 39-61 plaques for Gfap were analyzed per experimental group. For quantifying the percentage of Apoe⁺ or Serpina3n⁺ area in Gfap⁺ cells, 39-61 plaque regions for Apoe and 30-44 plaque regions for Serpina3n per experimental group were analyzed. The automatic thresholding methods in Fiji were used to quantify the positive areas (“Otsu/Otsu” for Apoe/Gfap; “RenyiEntropy/Triangle” for Serpina3n/Gfap). The overlay regions were calculated using customized macros in Fiji. To calculate the percentage of Plin2 in Iba1+ cells, The area of the Iba1+ cells and Plin2+ were selected by freehand selection and measured in Fiji. 8-24 cells per group were analyzed. To quantify the HJ3.4B⁺ plaque load and Lamp1 area, 10× magnification images were taken from the cortex of APP/PS1:APOE3-KI, APP/PS1:APOE3-cKO, APP/PS1:APOE4-KI, and APP/PS1:APOE4-cKO mice. The positive area of HJ3.4B and Lamp1 were quantified using automatic thresholding methods in Fiji with “Otsu” and “Triangle” separately. The average positive area was calculated for each animal. The number of plaques were quantified using customized macros in Fiji. 3-6 images per animal were taken, and 7-14 mice were quantified per group. To quantify the GFAP and pSMAD3 in human brain, 40× magnification images were taken from the cortical section of human AD subjects carrying APOE e3/3 and APOE e3/4 alleles. For GFAP quantification, individual plaques were selected and cropped for analysis in Fiji. The positive area of GFAP per plaque was quantified using the automatic thresholding method “Otsu” in Fiji. 43-52 plaques per group were analyzed. For pSMAD3 quantification, individual IBA1⁺ cells were selected and cropped for analysis in Fiji. The positive area of pSMAD3 per Iba1⁺ cell was quantified using the automatic thresholding method “Otsu” in Fiji. The positive area of pSMAD3 was further normalized to the Iba1⁺ cell area, and 27-33 cells per group were analyzed. To quantify the fluorescence of INPP5D in IBA1⁺ cells and ITGB8 in GFAP⁺ cells, 2-3 images per sample were collected in the gray matter. The automatic thresholding methods in Fiji were used to quantify the positive areas (“RenyiEntropy/Triangle” for INPP5D/IBA1; “Otsu/Triangle” for ITGB8/GFAP). The overlay regions were calculated using customized macros in Fiji. 7-8 samples per sex per group were analyzed for INPP5D, and for ITGB8, 7-8 male samples per group were analyzed. To quantify the fluorescence of Itgb8 in Gfap⁺ cells, 3-4 images per sample were collected in the cortex of APP/PS1:APOE4-KI and APP/PS1:APOE4-cKO mice. Itgb8⁺ positive areas in Gfap⁺ astrocytes were quantified using automatic thresholding methods in Fiji with “RenyiEntropy/Triangle” for Itgb8/Gfap. The overlay regions were calculated using customized macros in Fiji. 3-5 mice per group were analyzed. To measure the immunoreactivity of Clec7a and Gfap, 20× magnification images were taken from the cortex of control and Itgb8-cKO mice. The integrated density was measured in Fiji. Twelve ROIs per group were analyzed. The images were exported to Fiji for analysis. The positive area of HJ3.4B was quantified using the automatic thresholding method “Intermodes” in Fiji. 8-9 ROIs from 5 mice were analyzed per experimental group. The positive areas were quantified using the automatic thresholding method with “Moments” for Gfap and “RenyiEntropy” for HJ3.4B and Clec7a in Fiji. Eight ROIs from 5 mice were analyzed per experimental group. To quantify the plaque load and MHC II area in the anti-ITGB8-antibody-injected APP/PS1 brain, 20× and 40× magnification images were taken from the cortical section close to the injection site. The images were exported to Fiji, and the positive area of HJ3.4B or MHC II was quantified using the automatic thresholding method “Otsu” in Fiji. 4-7 mice per group were analyzed for MHC II. 8-9 mice per group were analyzed for HJ3.4B (two independent experiments combined, normalized to control group). To quantify the area of plaques, Clec7a and Gfap in the anti-ITGB8-antibody-injected APP/PS1:APOE4-KI brain, 20× and 40× magnification images were taken from the cortical section close to the injection site. The positive areas were quantified using the automatic thresholding method with “Otsu” for Clec7a, “Triangle” for HJ3.4B, and “RenyiEntropy” for Gfap in Fiji. Eleven ROIs from 6-7 mice per group were analyzed. To measure the load of Clec7a, Iba1, Lamp1, Tmem119, Gfap in the plaque area, 10× or 20× magnification images were taken from the cortex of APP/PS1 and APP/PS1:Inpp5d-cKO mice. The integrated density was measured using Fiji. 115-218 plaque regions for Clec7a and Iba1, 126-138 plaques for Lamp1 and Tmem119 and 127-154 plaques for Gfap per experimental group were analyzed. To quantify the HJ3.4B⁺ plaque load in the cortex, 10× magnification images were taken from the cortex of APP/PS1 and APP/PS1:Inpp5d-cKO mice. The positive area of HJ3.4B was quantified using the automatic thresholding method “Otsu” in Fiji. Seven mice (3 ROIs per mouse) were analyzed per experimental group.

### Statistical Analysis

Sample sizes for experiments were chosen based on prior publications using APP/PS1, P301S mice, and human analysis, which defined the expectations for the effect size and variance. Statistical analyses were performed using GraphPad Prism statistical software. All comparisons groups were assessed for normal distribution. We used Student's t-test for comparisons with only two groups and one-way ANOVA with Fisher's LSD post hoc test for multiple groups.

### Example 1. APOE4 Impairs Microglial Response to Acute Neurodegeneration

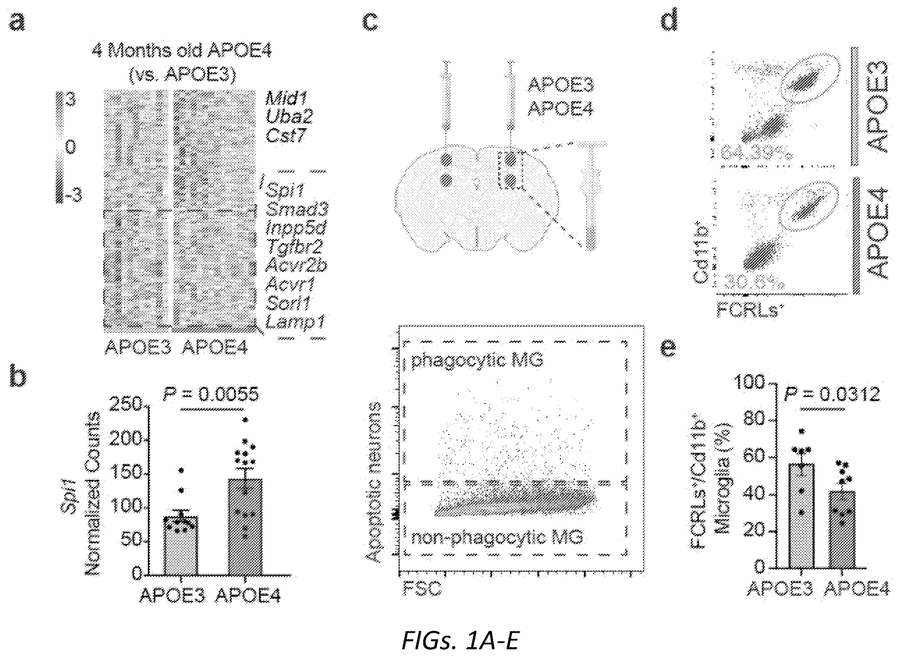

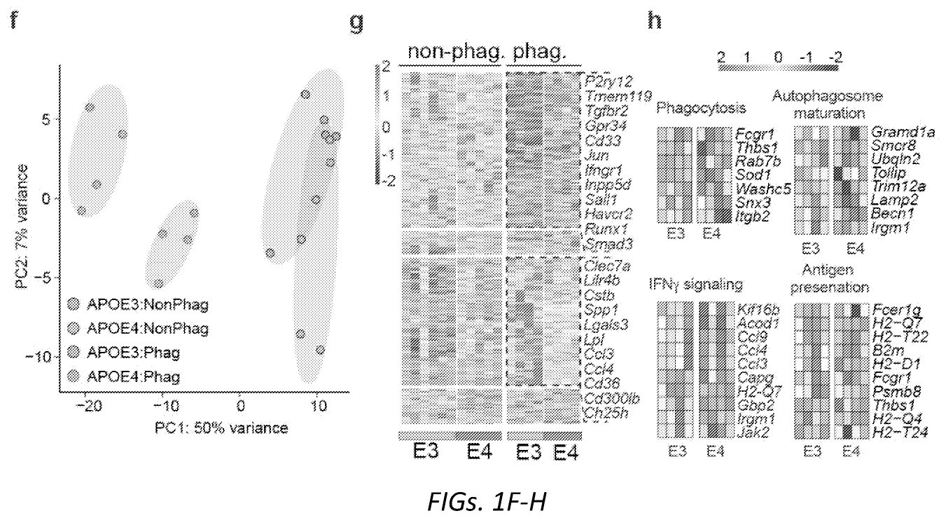

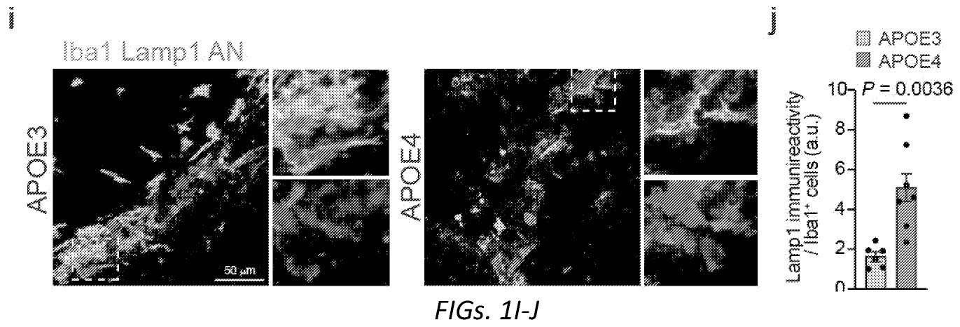

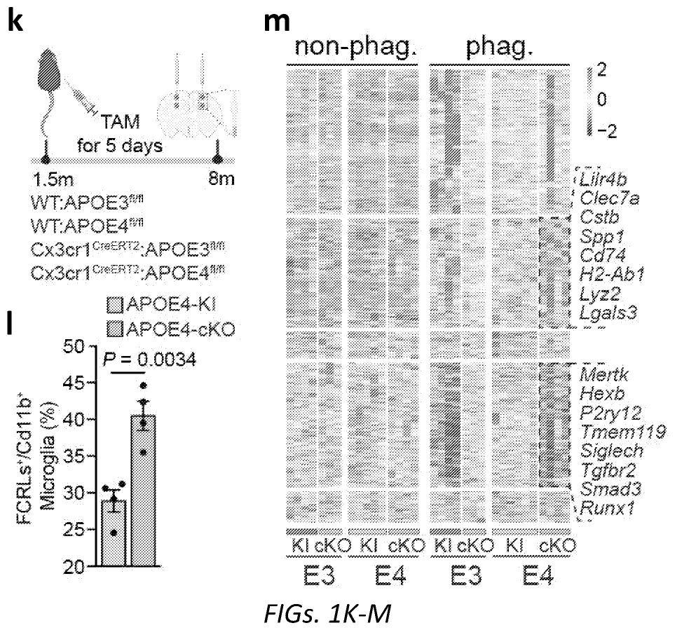

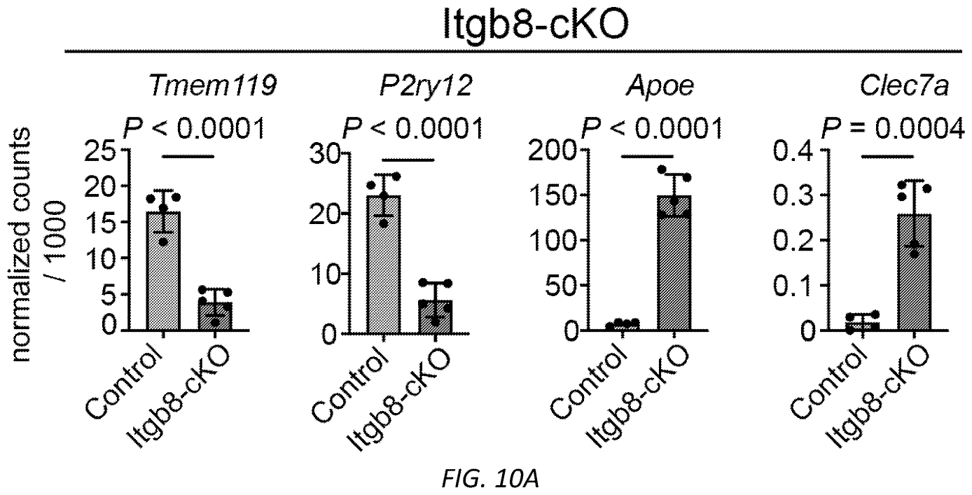

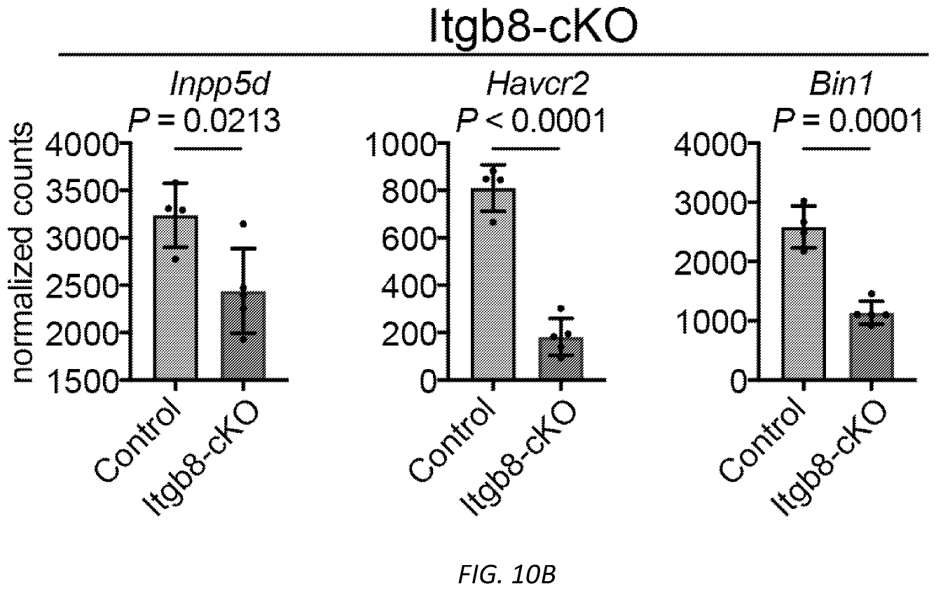

We recently showed that expression of APOE by microglia is required for the microglial phenotype switch from the homeostatic microglia, which is regulated by TGFb-signaling²¹, to MGnD in neurodegeneration⁶. To study the role of APOE variants in the regulation of microglial signature, we sorted microglia from APOE3-KI and APOE4-KI naïve mice at 4 months of age. Bulk RNA sequencing (RNAseq) analysis of isolated microglia identified significant upregulation of homeostatic genes in APOE4 microglia compared to APOE3 microglia at 4 months of age, including Tgfbr2, Inpp5d, Spi1 and Smad3 (FIG. 1a, b). Epigenetic analysis of genomic DNA bound to lysine 9 acetylated histone 3 (H3K9ac) by anti-H3K9ac chromatin immunoprecipitation (ChIP) sequencing analysis of isolated microglia revealed that APOE4 microglia displayed reduced chromatin acetylation as compared with APOE3 microglia. Furthermore, genomic regions associated with a homeostatic microglia signature, such as Inpp5d, Havcr2 and Smad3 showed enrichment for H3K9ac in APOE4 microglia compared to APOE3 microglia. To study the effect of APOE variants on the microglial response to acute neurodegeneration, we injected apoptotic neurons into the brains of APOE3 and APOE4 KI mice and sorted phagocytic and non-phagocytic microglia from the injection sites (FIG. 1c). FACS analysis of the injection site using the microglia specific marker Fcrls^{6, 21, 22} demonstrated impaired phagocytosis of dead neurons by Fcrls⁺/CD11b⁺ APOE4 microglia compared with APOE3 microglia (FIG. 1d,e). Using bulk RNAseq analysis of isolated microglia, APOE3 phagocytic microglia show induction of MGnD profile as compared with non-phagocytic microglia isolated from the same mice (FIG. 1f,g). APOE4 phagocytic microglia, however, failed to induce MGnD response of key genes, including Clec7a, Itgax, Lilr4b, Lpl, and Spp1 (FIG. 1g). Moreover, APOE4 microglia failed to upregulate the expression of genes associated with phagocytosis, antigen presentation, interferon-gamma (IFNg) signaling, and autophagosome maturation in response to phagocytosis of apoptotic neurons (FIG. 1h). Ingenuity pathway analysis (IPA) comparing phagocytic to non-phagocytic APOE3 and APOE4 microglia revealed dysfunctional response of APOE4 microglia to phagocytosis of dead neurons and antigen presentation. APOE4 was shown to promote accumulation of enlarged late endosomes in phagocytic cells²³ and in microglia²⁴, leading to impaired amyloid plaque clearance in AD-transgenic mice²⁵. Thus, we performed immunostaining of the microglia in the injection site and detected accumulation of Lamp1⁺ lysosomes in Iba1⁺ APOE4 microglia compared to APOE3 microglia (FIG. 1i, j). Furthermore, APOE4 microglia are impaired to respond to acute neurodegeneration, exhibiting reduced phagocytic Iba1⁺ cell numbers in the injection site compared to APOE3 microglia (FIG. 1i). To address the cell-autonomous role of APOE4 expressed in microglia in the regulation of microglial phenotype and functions, we crossed Cx3cr1^{CreERT2} mice²⁶ with APOE3-KI^{fl/fl} (APOE3-cKO) and APOE4-KI^{fl/fl} (APOE4-cKO) mice, which were recently described and used to study the role of APOE variants expressed by astrocytes in the regulation of neurodegeneration as well as multiple cellular phenotypes in tauopathy²⁰. APOE3-KI^{fl/fl} and APOE4-KI^{fl/fl} mice expressing Cx3cr1^{CreERT2/WT} and Cx3cr1^{WT/WT} gene were treated with tamoxifen (TAM) at the age of 1.5 months to conditionally delete APOE variants in microglia. Mice were injected with apoptotic neurons in the cortex and hippocampus at the age of 8 months (FIG. 1m). FACS analysis of cells isolated from the injection site showed increased recruitment of Fcrls⁺/Cd11b⁺ microglia in APOE4-cKO mice, as compared with APOE4-KI mice (FIG. 1n). Conditional deletion of microglial APOE4 restored the expression of key MGnD genes and downregulation of homeostatic genes in response to phagocytosis of apoptotic neurons (FIG. 10).

### Example 2. APOE4 Impairs Microglial Response to Neurodegeneration Via PU.1

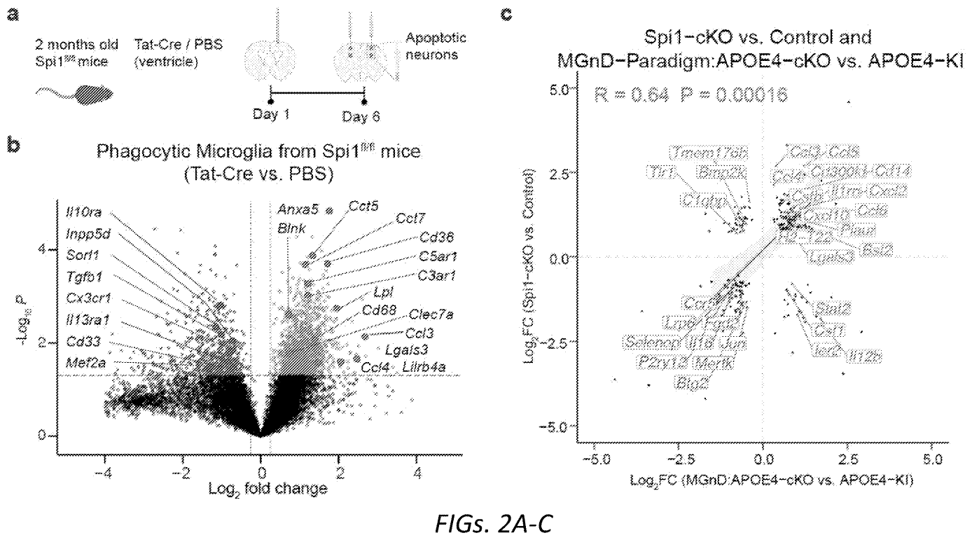

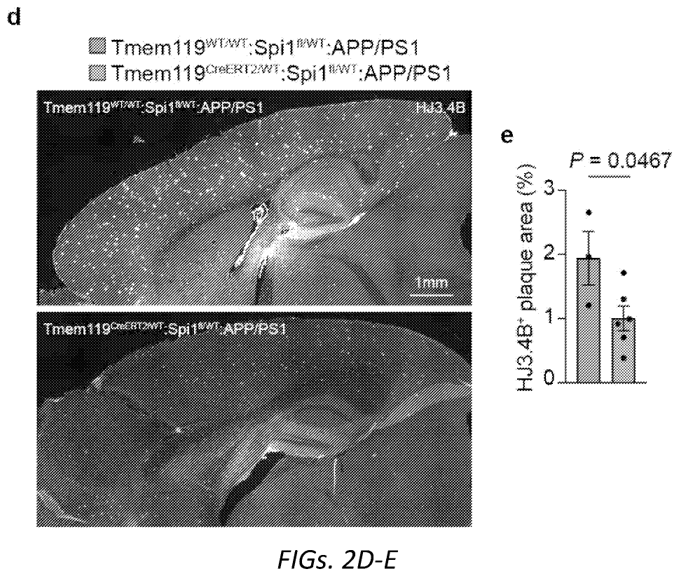

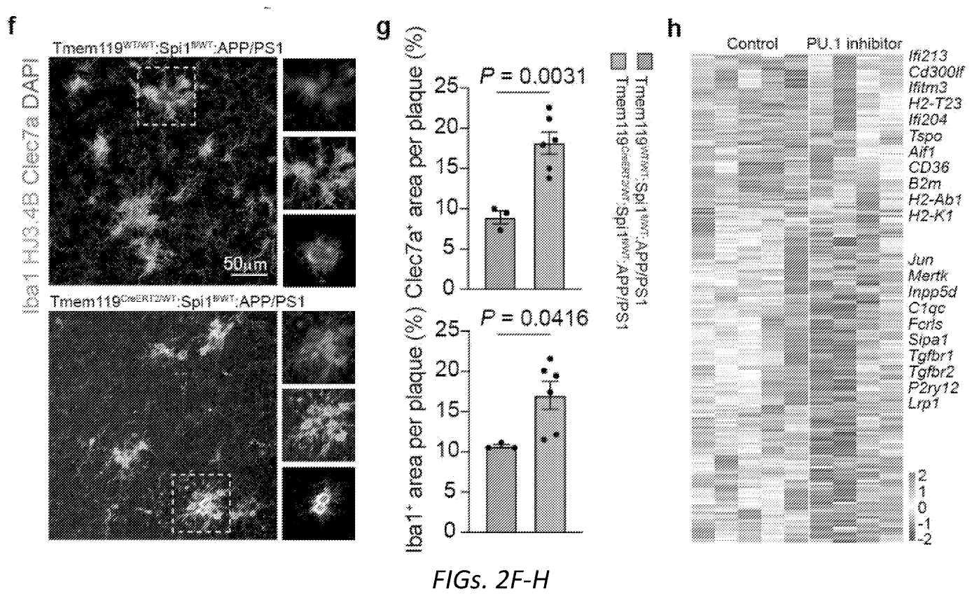

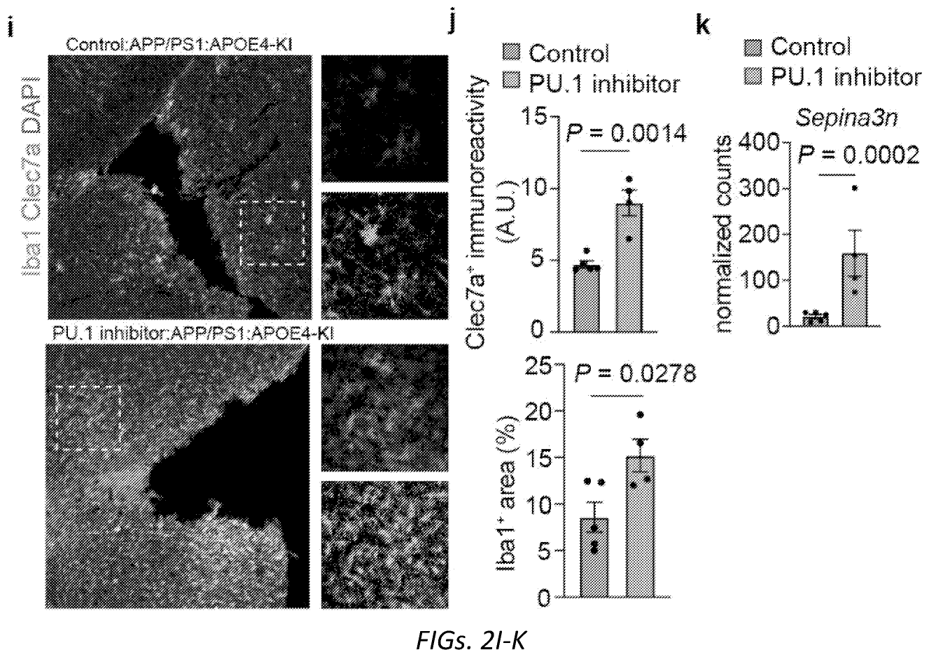

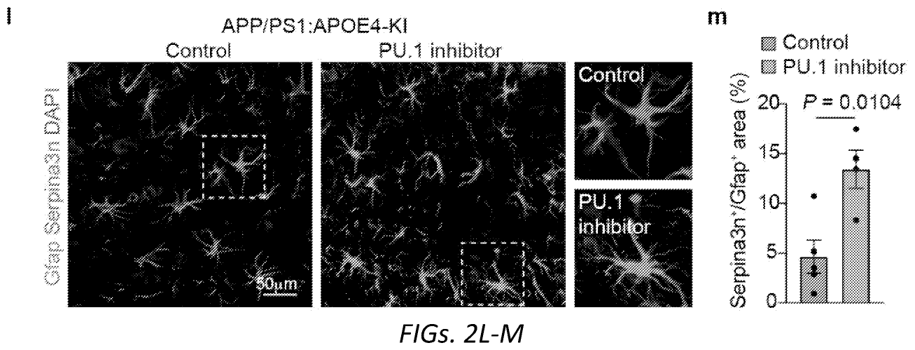

It was previously reported that the SMAD3 promoter was less acetylated in human AD prefrontal cortices as compared to controls²⁷. Spi1, also known as PU.1, binds SMAD3 to establish a homeostatic gene-regulatory-landscape in microglia²⁸. Importantly, low expression of PU.1 was suggested by a recent GWAS to be protective against AD²⁹. To address whether high expression of Spi1 in APOE4 microglia (FIG. 1a) maintains a homeostatic signature, we conditionally deleted microglial Spi1 in 2-month-old mice, which were subjected to intracranial injection of fluorescently labeled apoptotic neurons as a model of acute neurodegeneration⁶ (FIG. 2a). Deletion of Spi1 in microglia enhanced expression of MGnD genes including Clec7a, while homeostatic genes were downregulated including Tgfb1, Cd33, and Inpp5d (FIG. 2b). Moreover, by comparing Spi1 deletion and APOE4 deletion in phagocytic microglia (FIG. 10), we identified common MGnD and homeostatic genes induced and downregulated respectively in both conditions (FIG. 2c). To validate the enhanced MGnD response to amyloid pathology, we crossed

APP/PS1: Tmem119^{CreERT2} to Spi1^{fl/WT} mice to evaluate plaque pathology at 4 months of age after tamoxifen administration at 1.5 months of age (FIG. 2d,e). Microglial Spi1 deletion in APP/PS1 mice resulted in reduction in plaque load and enhanced Clec7a⁺ MGnD around amyloid plaques (FIG. 2d-g). This is further validated by RNAseq analysis of microglia isolated from APP/PS1:APOE4-KI mice treated with PU.1 pharmacological inhibitors, demonstrating an induction of the MGnD signature and suppression of homeostatic genes (FIG. 2h). Immunohistochemical analysis of mouse brains showed enhanced Clec7a⁺ MGnD response and increased astrocytic activation as determined by Serpina3n expression at around amyloid plaques (FIG. 2i-m). Taken together, these findings indicate that APOE4 predisposes microglia towards a homeostatic signature that interferes with microglial response to acute neurodegeneration and that targeting microglial APOE4 or PU.1 can restore MGnD induction.

### Example 3. Deletion of Microglial APOE4 Restores MGnD Response to Chronic Neurodegeneration and Promotes Neuroprotection

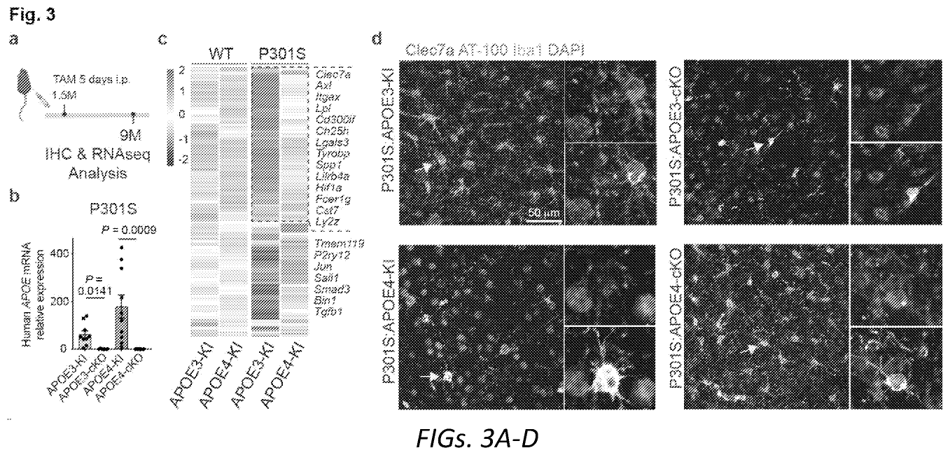

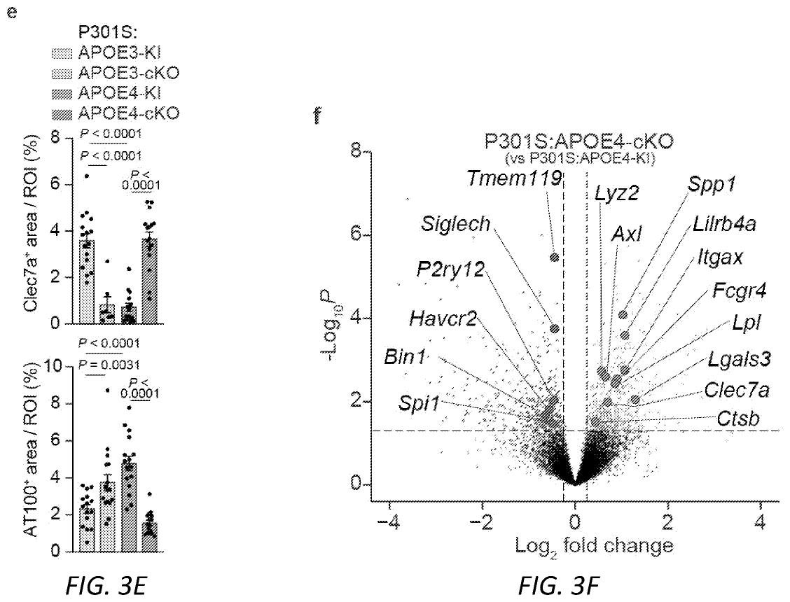

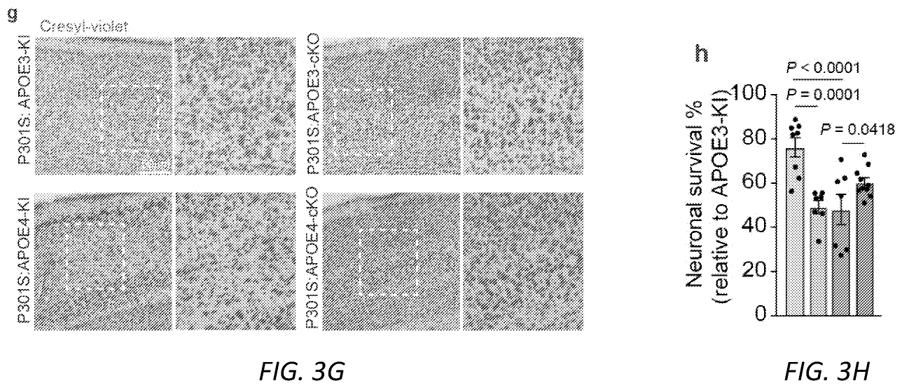

Neurodegeneration is a pathological hallmark of AD pathology³⁰, which is exacerbated in a mouse model of tauopathy expressing APOE420, 31. To dissect the impact of microglial APOE variants on tau-induced neurodegeneration, Cx3cr1^{CreERT2/WT}:APOE3-KI^{fl/fl} and Cx3cr1-CRE^{ERT2/WT}:APOE4-KI^{fl/fl} mice were crossed to P301S mice, which develop tau pathology and neurodegeneration between 6 to 9.5 months of age³². These mice were treated with tamoxifen at the age of 1.5 months and evaluated at the age of 9 months (FIG. 3a). qPCR analysis confirmed the deletion of the human APOE gene in microglia expressing Cx3cr1^{CreERT2} (FIG. 3b), with no significant difference in APOE immunoreactivity comparing P301S:APOE3-KI and P301S:APOE4-KI. Microglia sorted from the brains of P301S:APOE3-KI mice at 9 months of age upregulated key MGnD genes including Clec7a and Itgax, while downregulating TGFb-signaling molecules including Smad3 and Tgfb1, as compared to microglia isolated from WT:APOE3-KI mice (FIG. 3c). In contrast, microglia from P301S:APOE4-KI mice showed reduced MGnD and increased TGFb-signaling signature compared to microglia from age-matched P301S:APOE3-KI mice (FIG. 3c). Immunohistochemical analysis confirmed decreased Clec7a immunoreactivity in cortical microglia of P301S:APOE4-KI mice, which exhibited increased tau hyperphosphorylation as compared to age-matched P301S:APOE3-KI mice (FIG. 3d,e). In previous studies, brain regions of P301S:APOE4-KI mice demonstrate marked neurodegeneration compared to P301S:APOE3-KI mice at 9.5 months of age, although the MGnD phenotype was similar between the two groups^{20, 31} We found that conditional deletion of APOE4 in microglia was sufficient to restore Clec7a immunoreactivity and the MGnD expression signature in P301S mice, which was associated with reduced tau hyperphosphorylation (FIG. 3d-f). To determine the effect of microglial deletion of APOE variants on neurodegeneration, we quantified neurons stained with Cresyl Violet in cortical layer 5, as previously described in tau mice³³. P301S:APOE4-KI showed increased neuronal loss in cortical layer 5 compared with P301S:APOE3-KI, whereas neuronal loss was significantly reduced in P301S mice deleted for microglial APOE4 compared to age-matched P301S:APOE4-KI mice (FIG. 3g,h). These results suggest that microglial expression of APOE4 is important for MGnD response to tau-mediated neurodegeneration in cortex and that its deletion in microglia delayed neuronal loss.

### Example 4. Microglial Deletion of APOE4 Restores MGnD-Microglia and Facilitates Ab Plaque Clearance in APP/PS1 Mice

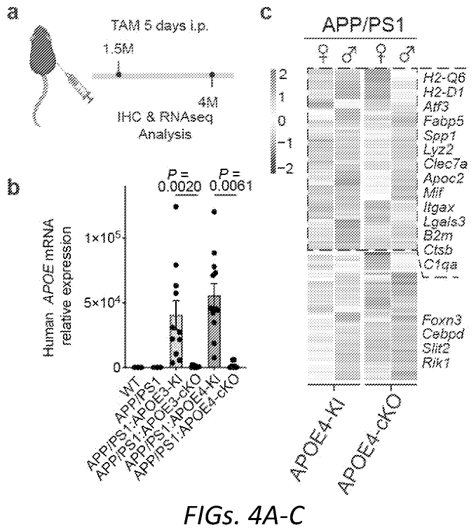

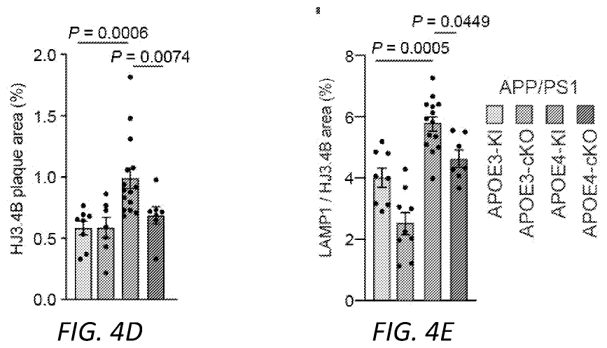

APOE4 was shown to promote Ab plaque pathology in both mouse models and in AD brains^{8, 9, 10, 11, 12, 13}. To investigate the cell-intrinsic regulation of APOE4 in microglia, we used the APP/PS1 mouse model, which develop plaque pathology as early as 2 months of age³⁴ crossed to Cx3cr1^{CreERT2/WT}:APOE3-KI^{fl/fl} and Cx3cr1^{CreERT2/WT}:APOE4-KI^{fl/fl} mice, referred as APP/PS1:APOE3-cKO and APP/PS1:APOE4-cKO, respectively. These mice were treated with tamoxifen at the age of 1.5 months and evaluated at the age of 4 months to validate the deletion of human APOE in microglia (FIG. 4a,b). RNAseq analysis of sorted microglia showed restoration of MGnD signature in APP/PS1:APOE4-cKO mice compared with APP/PS1:APOE4-KI mice (FIG. 4c). To characterize in more detail the microglial response in APP/PS1 mice we took advantage of single-cell RNAseq of brain cells isolated from APP/PS1:APOE4-KI and APP/PS1:APOE4-cKO mice. We applied unsupervised clustering and identified 6 clusters showing microglia-like expression profiles. To distinguish peripheral recruited myeloid cells and border associated macrophages (BAM) from microglia, we re-clustered all microglia-like cells and identified Ms4a7⁺ and Mrc1⁺ macrophages^{35, 36} and Lyve1⁺ BAM cells^{37, 38}. Out of all microglia, we identified 3 major subtypes, which we labelled as 1) M0 (homeostatic microglia), expressing Tmem119, P2ry12 and Gpr34, 2) MGnD, expressing Clec7a and Spp1, and 3) Interferon-microglia enriched for Stat1, Irf7, and Ilfit1 expression. The restoration of an MGnD phenotype in APP/PS1:APOE4-cKO was accompanied by a significant reduction in HJ3.4B⁺ Aβ plaque pathology (FIG. 4D) and Lamp1⁺ dystrophic neurites (FIG. 4E). Notably, the immunoreactivity of Lamp1⁺ dystrophic neurites was enhanced in microglia deleted from APOE3 (FIG. 4E). APOE4 microglia derived from induced pluripotent stem cells were shown to accumulate lipid droplets that disrupts the coordinated microglia-neuron crosstalk, required to support the homeostasis of neuronal ensembles³⁹. To study whether APOE4-KI microglia display altered metabolic profile, we performed lipidome analysis of sorted microglia from mice harboring APOE variants on APP/PS1 and WT background. We detected increased levels of certain lipid species in APOE4-KI microglia compared with APOE3-KI microglia, which were more pronounced on APP/PS1 background. Moreover, a recent study identified APOE4-driven lipid metabolic dysregulation in human astrocytes and microglia²⁴, which may be exacerbated by AD pathology. Immunohistochemistry confirmed the increased accumulation of Plin2 lipid droplets in microglia in APP/PS1:APOE4-KI mice, which was reduced following the conditional deletion of APOE4 in microglia. Collectively, these results suggest a beneficial role of MGnD microglia in limiting AD pathology, that is disrupted by APOE4 and its deletion in microglia is sufficient to restore MGnD response to phagocytic stress and neurodegeneration.

### Example 5. Microglial APOE4 Deletion Promotes Astrocyte Activation and Encapsulation of Ab Plaques Via Lgals3 Signaling

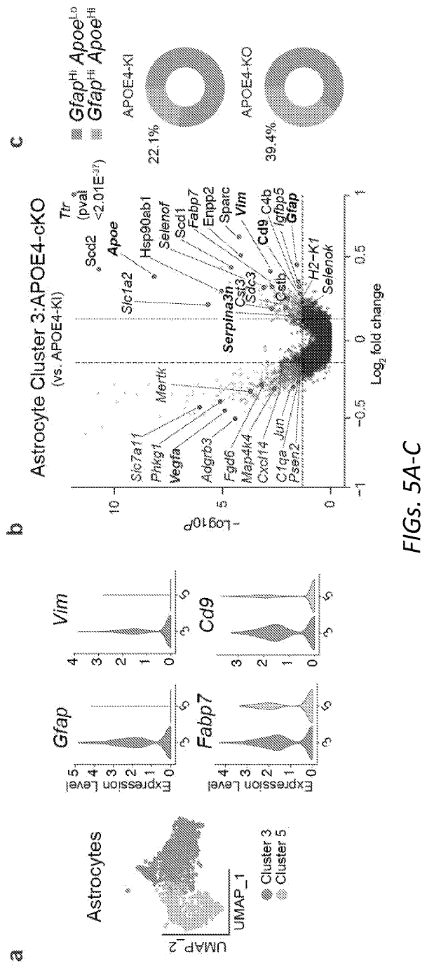

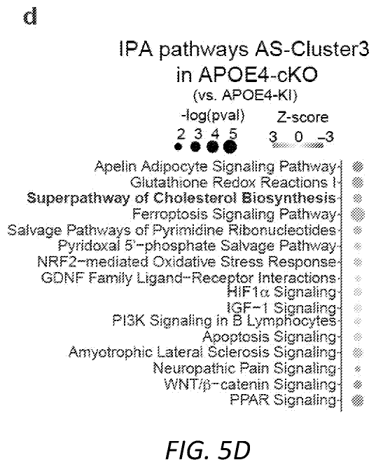

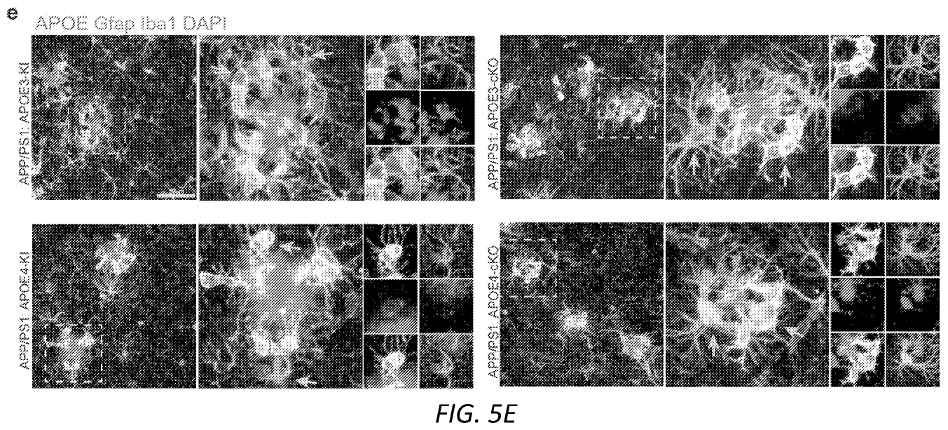

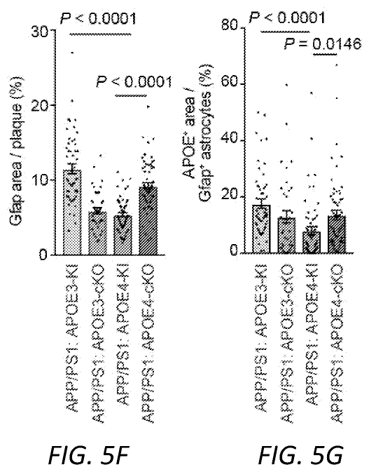

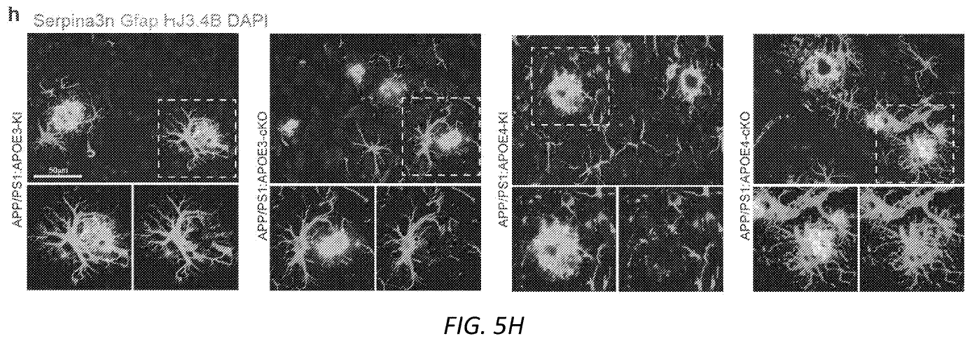

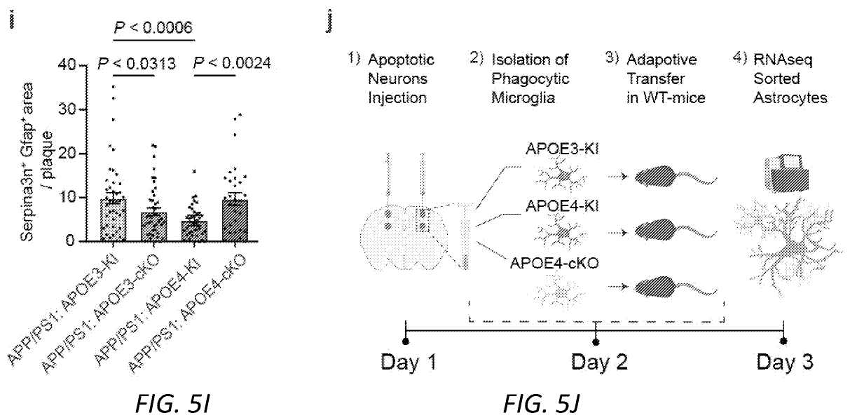

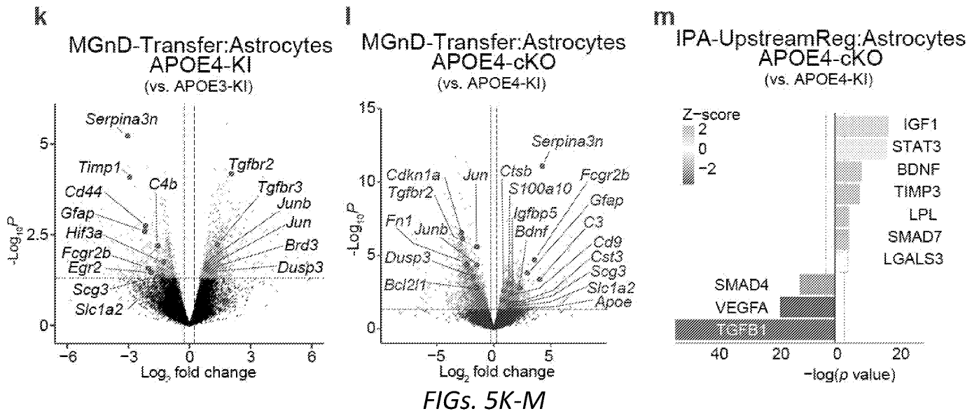

Astrocytic expression of APOE4 was shown to associate with metabolic dysfunction and to contribute to tau pathology²⁰. However, the impact of microglial APOE4 on astrocyte phenotype and function is unknown. Using scRNAseq analysis of brain cells isolated from APP/PS1:APOE4-KI vs. APP/PS1:APOE4-cKO mice, we detected two astrocyte clusters (3 and 5). Cluster 3 was enriched for key reactive astrocyte genes, including Gfap, Vim, Fabp7, Cd9, and Serpina3n, as compared to Cluster 540 (FIG. 5a). Deletion of microglial APOE4 further induced expression of Apoe, Ttr, Cd9, Vim, Gfap, and Serpina3n in Cluster 3 (FIG. 5b). The top induced gene in Cluster 3 was Ttr (FIG. 5b), an APOE transporter implicated in disrupting Ab fibril formation⁴¹. Of note, in APP/PS1:APOE4-cKO, Cluster 3 astrocytes significantly downregulated Vegfa (FIG. 5b), which was shown to disrupt blood-brain barrier integrity⁴². Furthermore, microglial deletion of APOE4 increased the ratio of Gfap^{Hi}/Apoe^{Hi}, Gfap^{Hi}/Serpina3n^{Hi}, Gfap^{Hi}/Vim^{Hi}, and Gfap^{Hi}/Cd9^{Hi} astrocytes (FIG. 5c). IPA analysis of astrocytes from APP/PS1:APOE4-cKO mice showed upregulation of metabolic pathways of cholesterol biosynthesis, response to oxidative stress, HIF1a and IGF-1 signaling (FIG. 5d). IHC analysis showed decreased Gfap immunoreactivity around Ab plaques in APP/PS1:APOE4-KI mice as compared to APP/PS1:APOE3-KI mice, while APP/PS1:APOE4-cKO mice restored Gfap immunoreactivity (FIG. 5e-f). Immunostaining for human APOE protein and Gfap validated the deletion of APOE in Iba1⁺ microglia of APP/PS1:APOE3-cKO and APP/PS1:APOE4-cKO mice, despite the detection of APOE immunoreactivity in plaques and astrocytes (FIG. 5e). Moreover, APOE immunoreactivity in Gfap⁺ astrocytes was also restored in APP/PS1:APOE4-cKO mice (FIG. 5e,g), confirming results from scRNAseq analysis (FIG. 5b). Furthermore, protein expression of Serpina3n in Gfap⁺ astrocytes was reduced in APP/PS1:APOE3-cKO and APP/PS1:APOE4-KI mice as compared to APP/PS1:APOE3-KI mice (FIG. 5h,i), whereas deletion of APOE4 in microglia increased Serpina3a in Gfap⁺ astrocytes encapsulating Ab plaques (FIG. 5h).

To address direct microglia-astrocyte crosstalk affected by APOE3 vs. APOE4 alleles expressed in microglia, we transplanted MGnD-phagocytic microglia from APOE3-KI, APOE4-KI and APOE4-cKO donor mice injected with apoptotic neurons into WT naïve mice (FIG. 5j). Astrocytes were isolated from the injection sites 16 h later and analyzed by bulk RNAseq. We found reduced expression of astrocyte activation markers, including Serpina3n and Gfap in mice injected with APOE4-MGnD as compared to APOE3-MGnD (FIG. 5k). Importantly, deletion of APOE4 in microglia restored their crosstalk with astrocytes and induced classical astrocyte activation molecules including Serpina3n, Cd9, and Gfap (FIG. 5l). IPA analysis identified IGF-1 as the most activated upstream regulator, whereas TGFb1 was the top suppressed upstream regulator in astrocytes in response to APOE4-cKO MGnD (FIG. 5m). Taken together these findings support the critical role of MGnD-microglia expressing Lgals3 in promoting astrocytes activation and their recruitment to plaques.

### Example 6. Impaired Induction of MGnD Signature and Astrocyte Activation in APOE e4 AD Carriers

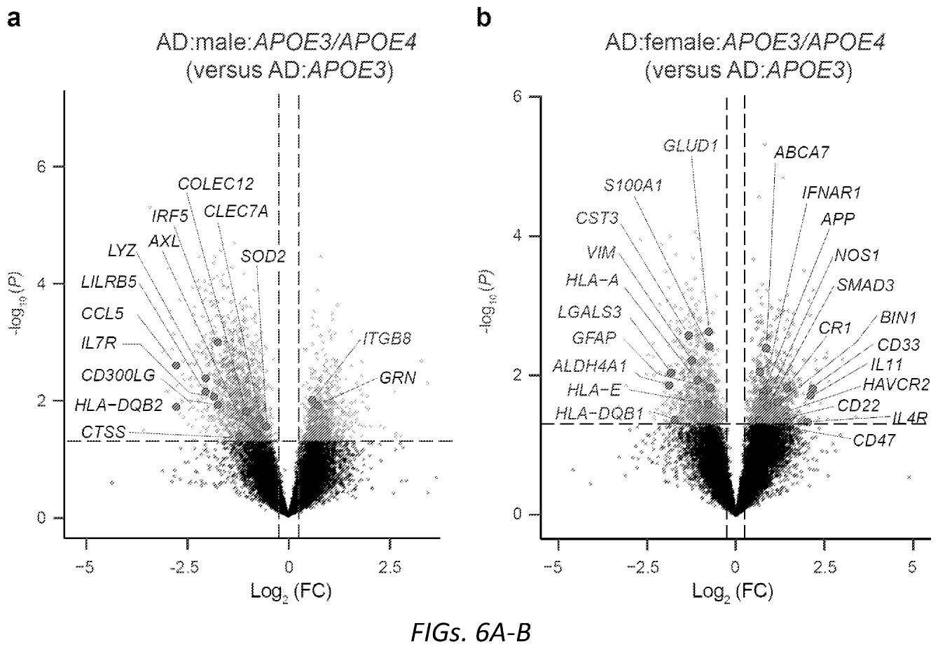

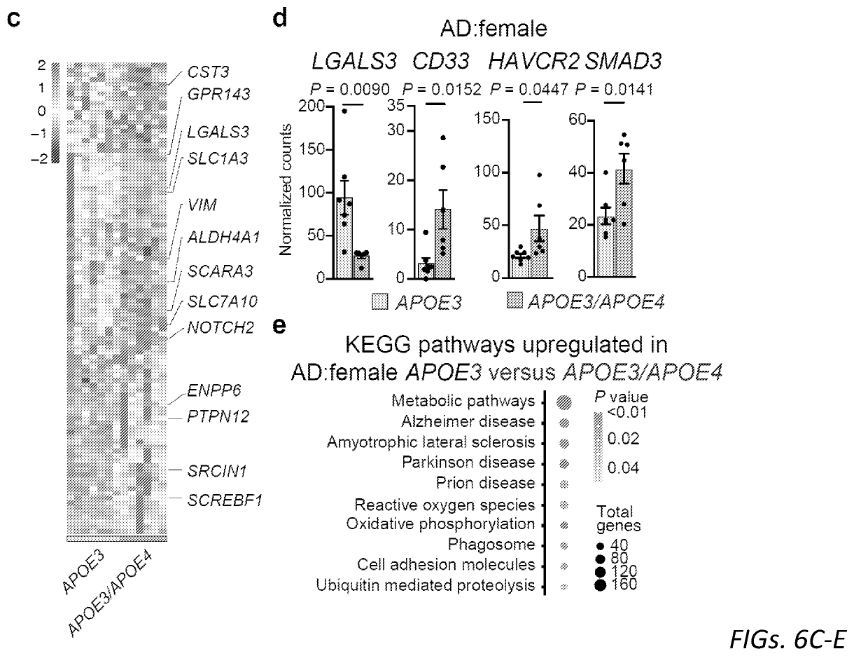

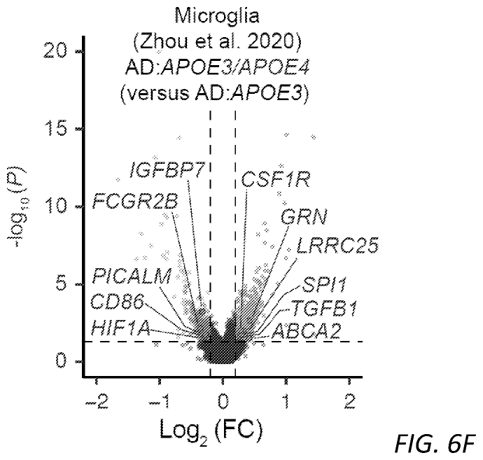

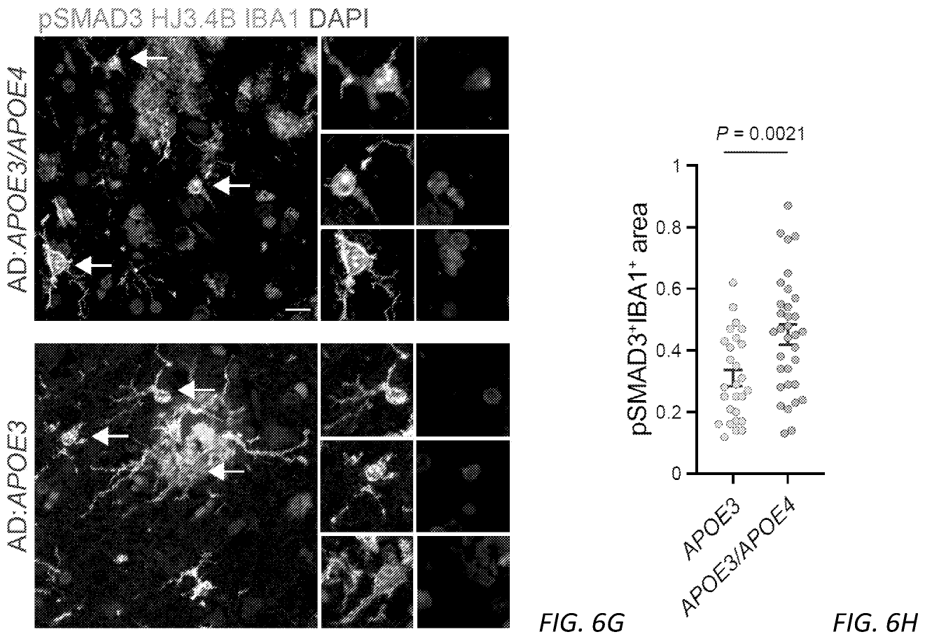

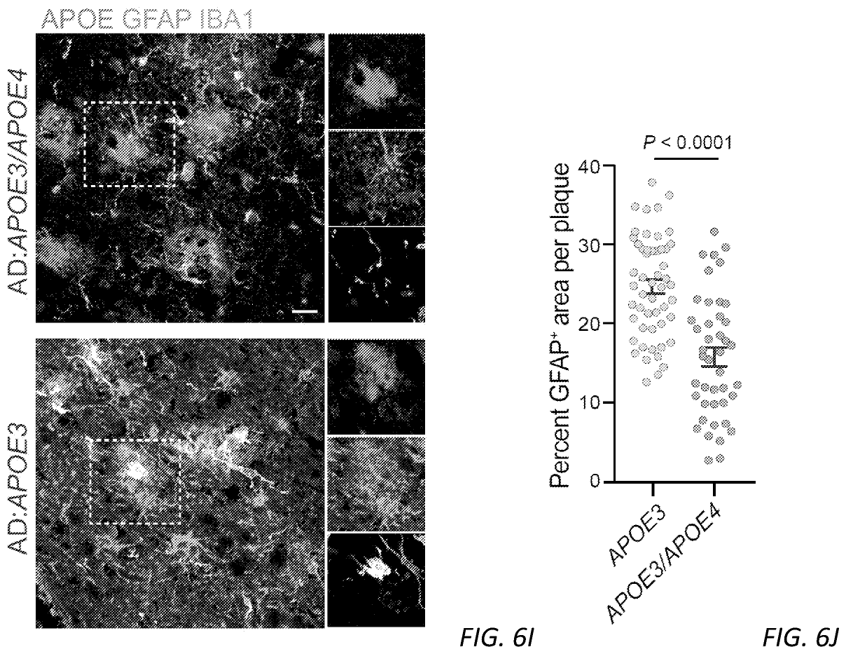

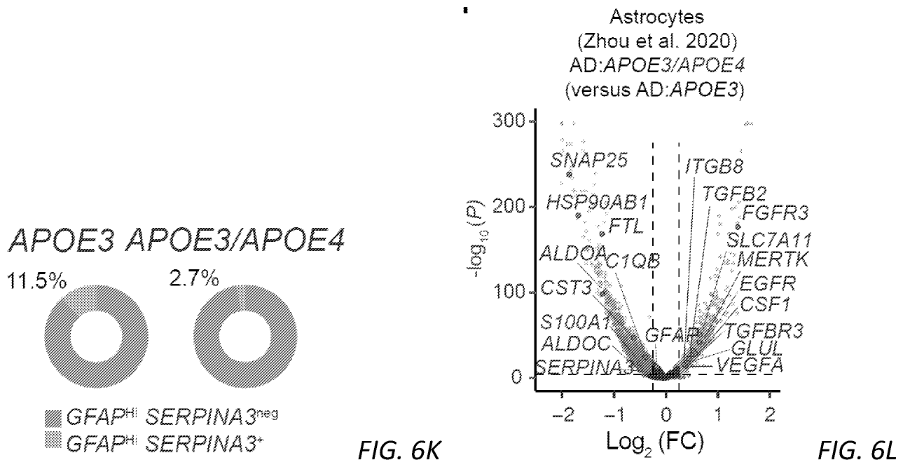

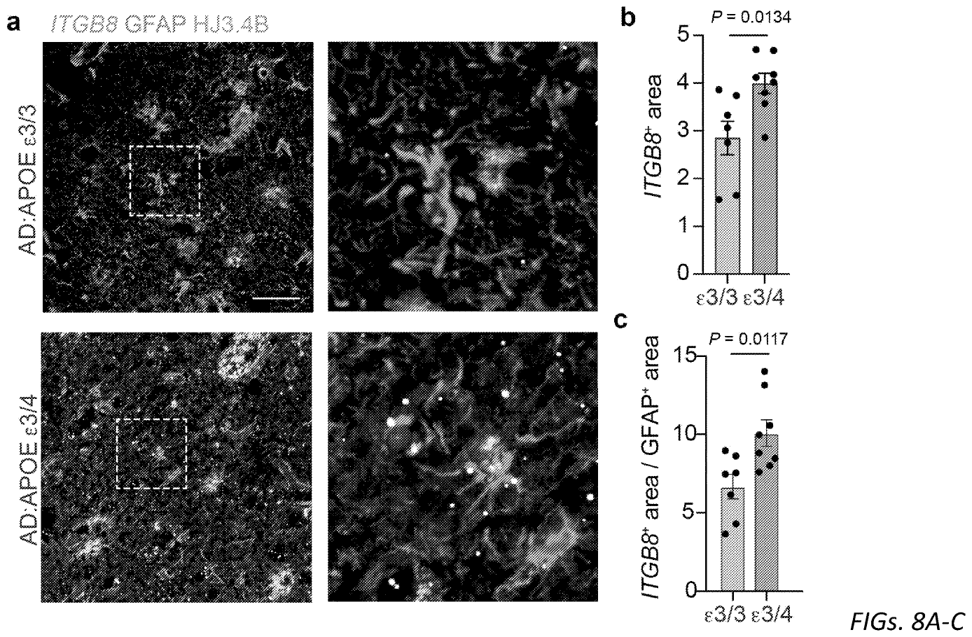

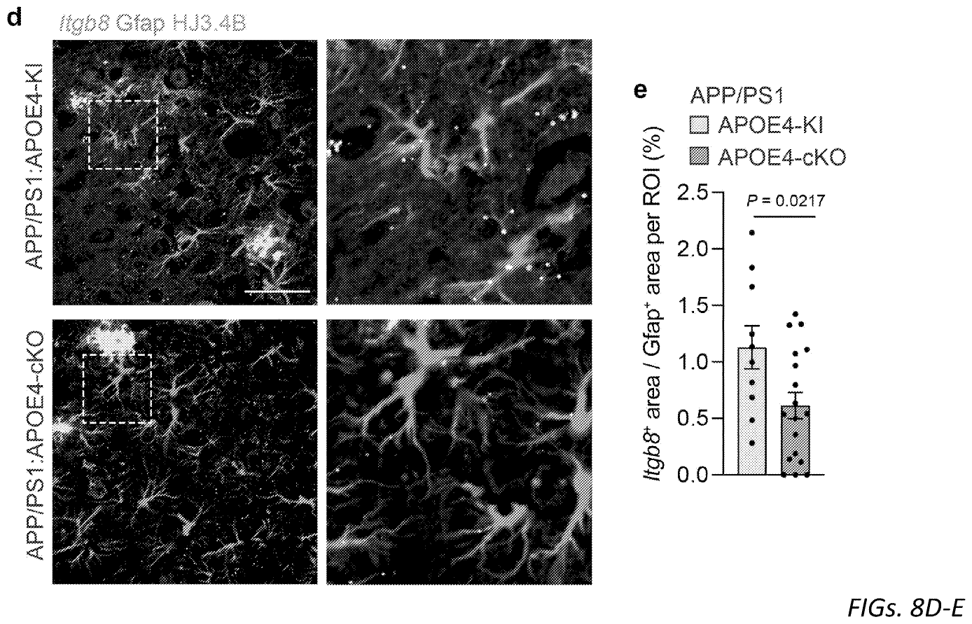

Women carrying the APOE4 allele are at greater risk of developing AD and show an accelerated disease compared with male carriers of APOE e4⁸. To study the sex differences related to APOE4 in AD subjects, we used RNAseq analysis of total human brains. In male AD brains with APOE e3/4 allele, there was a significant reduction in the expression levels of MGnD genes such as CLEC7A, AXL, LYZ, CD300LG and HLA-DQB2, whereas the upstream regulator of TGFb signaling ITGB8 was more induced compared to APOE e3/3 brains (FIG. 6a). We also found GRN expression was significantly increased in APOE e3/4 males, which was previously reported to correlate with reduced microglia activation in humans⁴⁵. Moreover, in line with reduction of astrocyte activation in APP/PS1:APOE4-KI mice, we found reduced expression of S100A, GFAP, and VIM in female APOE e3/4 AD brains (FIG. 6b,c). Importantly, among the top genes upregulated in female APOE e3/4 AD brains, were several AD risk factors including ABCA7, IFNAR1, APP, BINI, CD33 and HAVCR2 (FIG. 6b,d), which encodes the checkpoint molecule T-cell immunoglobulin mucin-3, one of the most promising new therapeutic approaches in cancer⁴⁶, and was recently described in GWAS as an AD risk gene⁴. In addition, SMAD3, a downstream adaptor of TGFb signaling⁴⁷ was upregulated in APOE e3/4 AD female brains (FIG. 6b,d). KEGG pathway analysis of female microglial RNAseq data showed enrichment of pathways associated with neurodegenerative diseases, reactive oxygen species, phagosome formation and proteolysis in APOE e3/3 AD carriers (FIG. 6e). Analysis of publicly available human brain scRNAseq data⁴⁸ showed increased expression of TGFb signaling genes and downstream AD risk factors including INPP5D in female APOE e3/4 carriers. Meanwhile, MGnD genes such as APOE, SPP1, and HLA-DOBI were reduced in female APOE e3/4 carriers ( ). Importantly, we found a similar observation in an independent cohort described by Zhou et al.⁴⁹ showing increased expression of SPI1, TGFB1 and GRN in microglia from AD brains expressing APOE e3/4 (FIG. 6f). Moreover, RNAscope analysis confirmed the increased expression of the homeostatic checkpoint INPP5D in IBA1⁺ microglia in APOE e3/4 AD carriers, as compared to APOE e3/3 AD carriers. These findings in humans are in line with our previous results demonstrating enriched Spi1 expression in APOE4-KI mice (FIG. 1a) and restoration of MGnD signature following genetic deletion of Spi1 (PU.1) in mice challenged with apoptotic neurons. PU.1 was shown to bind SMAD3 promoting the maintenance of microglial homeostatic signature⁵⁰. Importantly, we detected increased expression of SMAD3 in female APOE e3/4 brains (FIG. 6b, suggesting that PU.1 promotes TGFb regulation in APOE e3/4 AD carriers and support previous observation that higher SPI1 expression is likely to have an earlier AD onset²⁹. Immunohistochemistry analysis confirmed SMAD3 activation and pSMAD3 immunoreactivity in IBA1⁺ microglia in APOE e3/4 AD females (FIG. 6g,h). We also confirmed that GFAP immunoreactivity, associated with Ab plaques, was reduced in female APOE e3/4 brains as compared to APOE e3/3 brains (FIG. 6i,j). Analysis of public snRNAseq data⁴⁹ of astrocytes from AD brains expressing APOE e3/3 and e3/4 alleles confirmed that the percentage of GFAP^{Hi} and SERPINA3⁺ astrocytes is increased in APOE e3/3 donors as compared to APOE e3/4 donors (FIG. 6k). Furthermore, astrocyte activation genes including CST3, HSP90ABI and ALDOC were reduced, while TGFb1 signaling related genes including TGFBR3, TGFB2 and ITGB8 were significantly increased in APOE e3/4 subjects (FIG. 6l). To confirm the increase in ITGB8 expression levels in astrocytes, we performed RNAscope analysis and detected induction of ITGB8 in GFAP⁺ astrocytes in APOE e3/4 compared with APOE e3/3 AD males (FIG. 8A-Ci). Furthermore, RNAscope showed reduced Itgb8 expression in astrocytes from APP/PS1:APOE4-cKO mice compared to APP/PS1:APOE4-KI mice (FIG. 8D-E). Taken together, these findings show increased TGFb signaling in APOE4 carriers, which may predispose the development of AD via the inhibition of MGnD-microglia and astrocytes response to neurodegeneration.

### Example 7. Deletion of ITGB8-TGFb Signaling Enhances MGnD Response and Aβ Phagocytosis

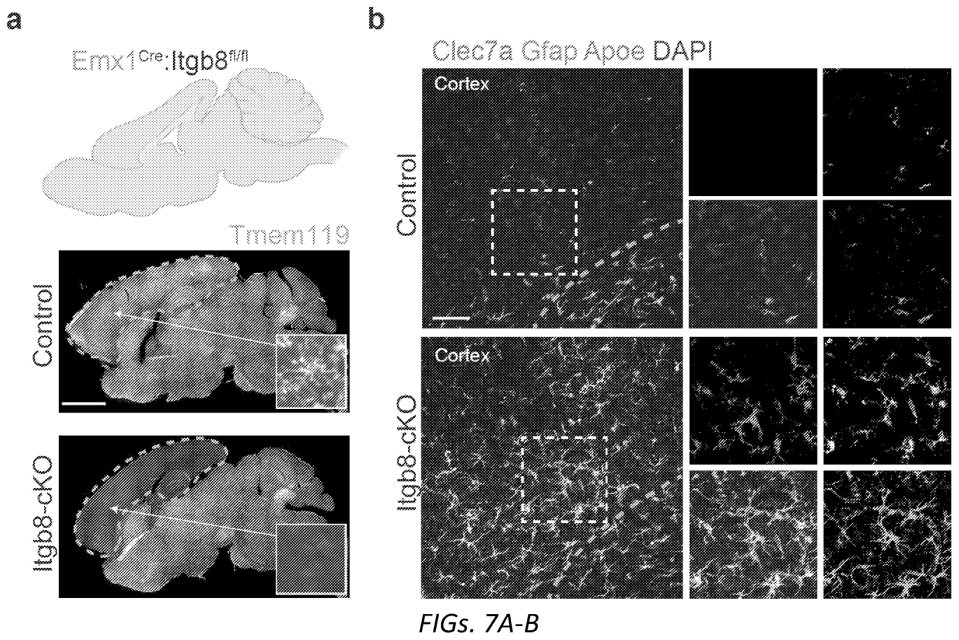

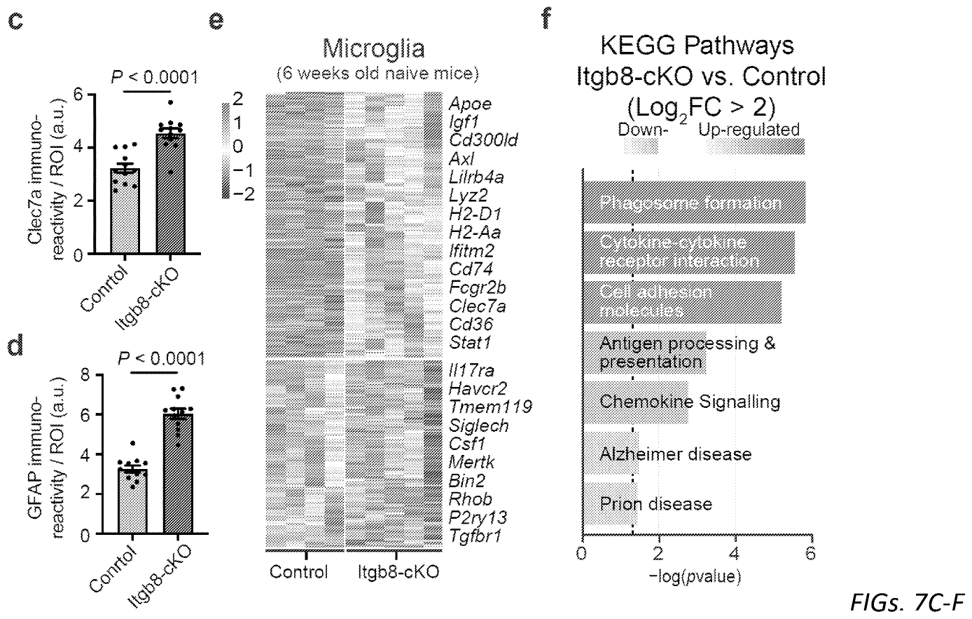

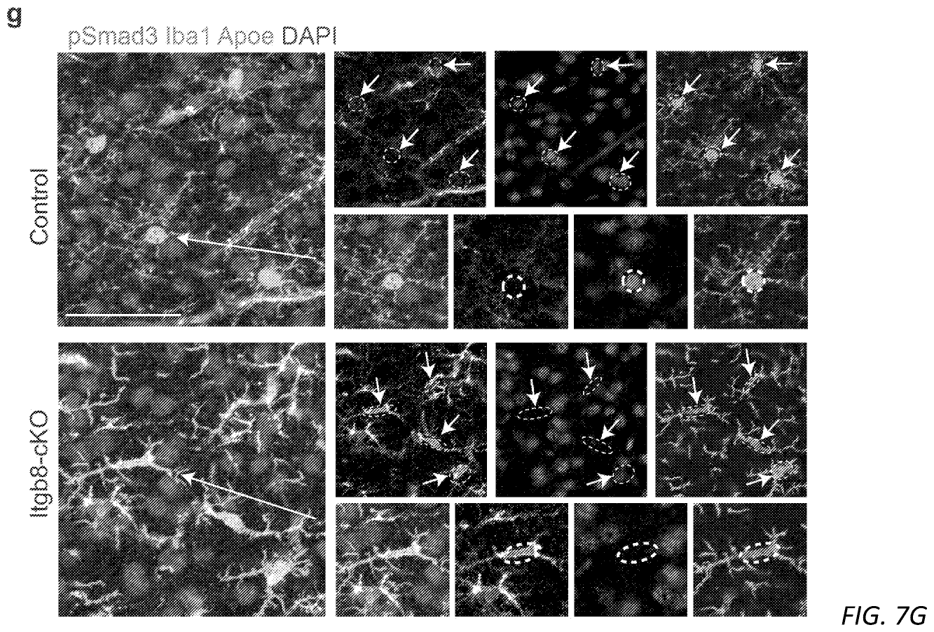

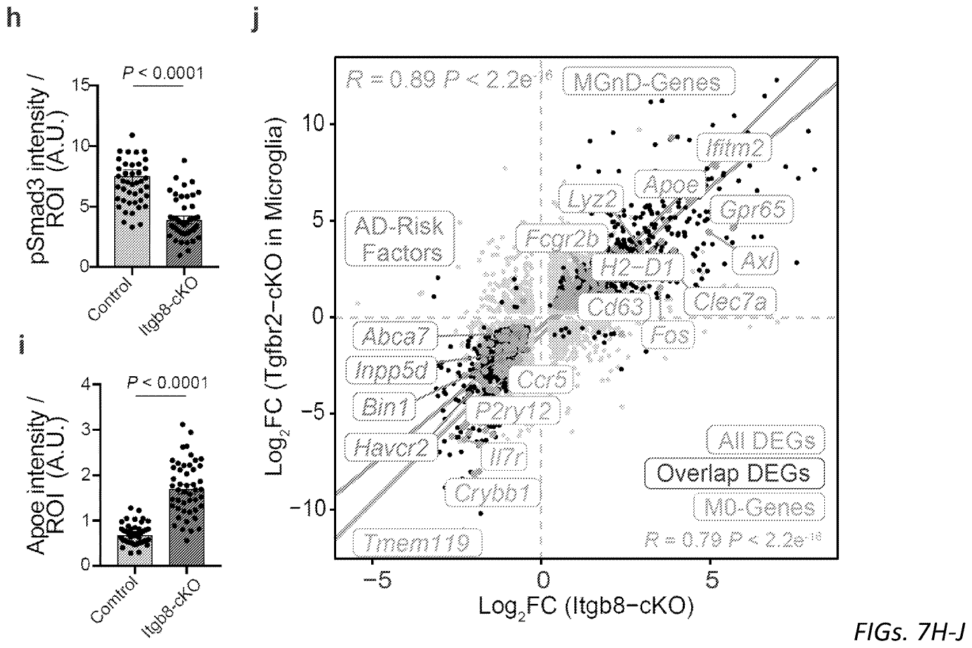

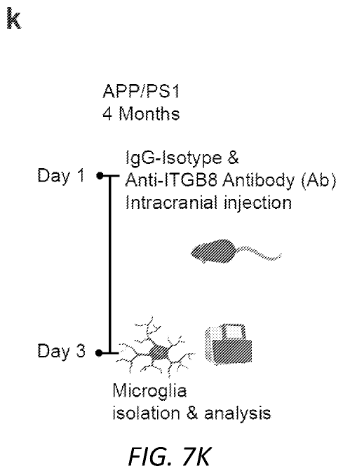

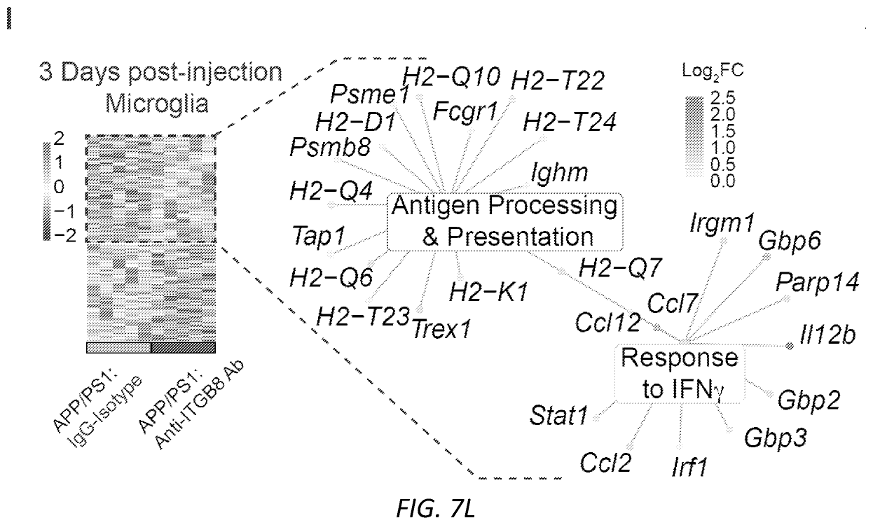

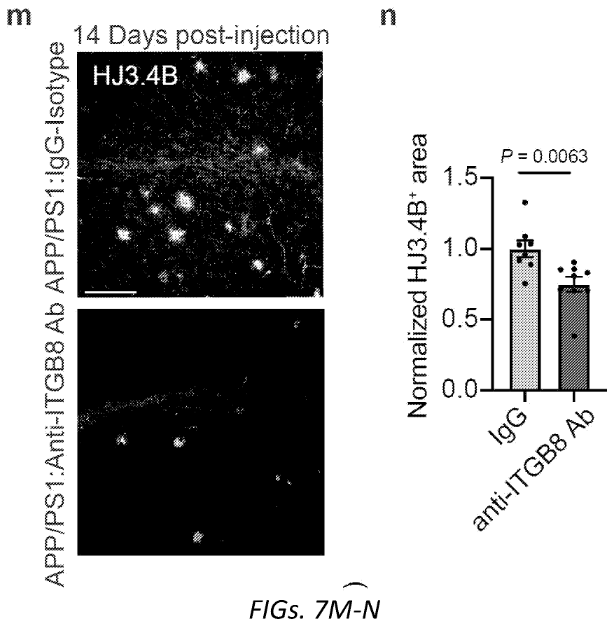

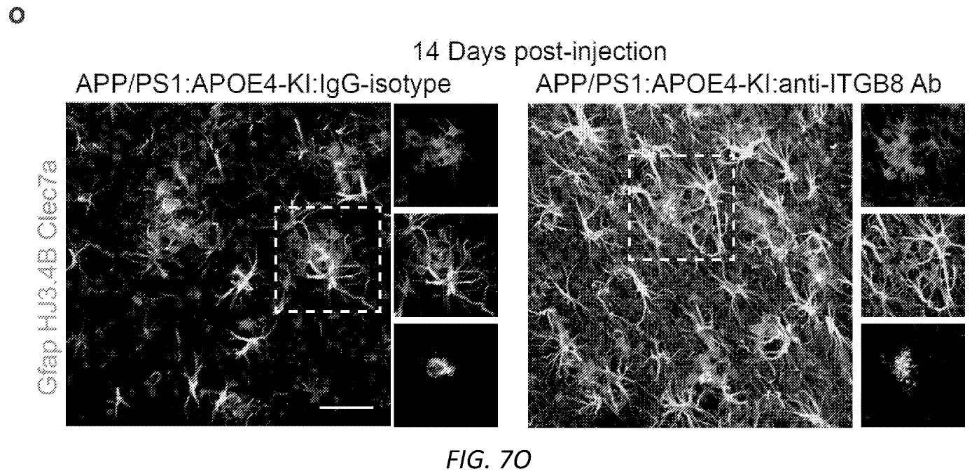

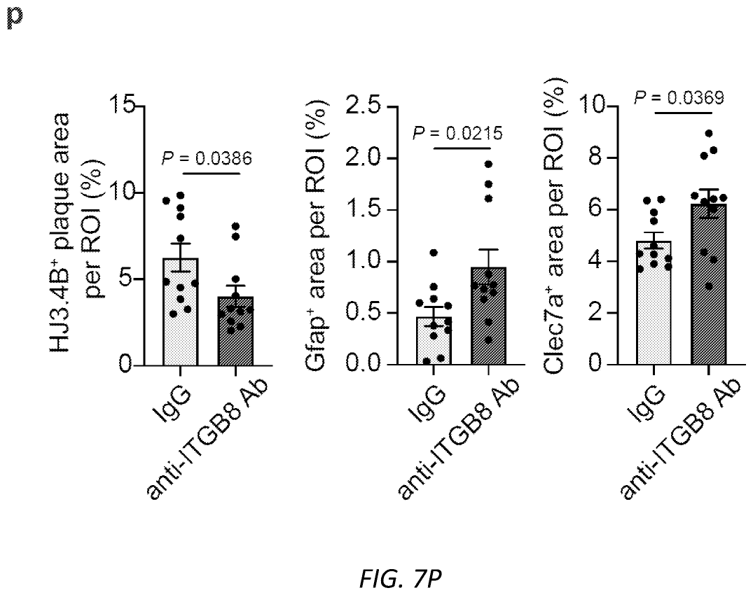

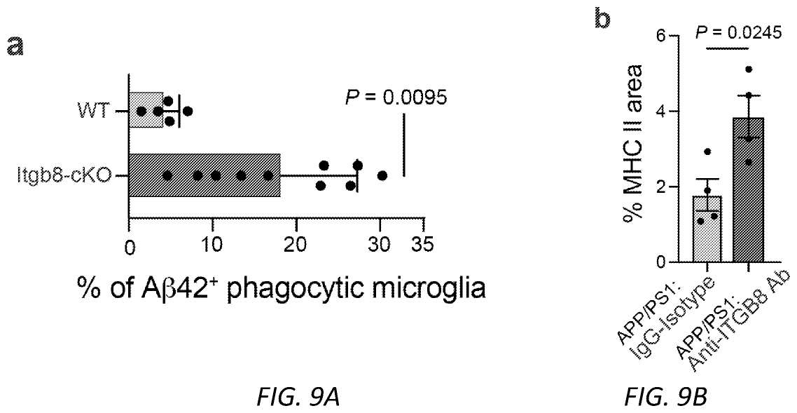

To gain molecular insights into the role of APOE4-mediated induction of TGFb signaling in AD brains, we genetically and pharmacologically inhibited ITGB8, which is critical for the activation of latent TGFb1⁵¹, in WT and APP/PS1 mice. Consistent with a published brain RNAseq dataset⁵², Itgb8⁻TdTomato reporter mice revealed strong expression of Itgb8 in cortical astrocytes, mature oligodendrocytes, and oligodendrocyte precursors cells, but not in microglia, neurons, or endothelial cells. Genetic deletion of Itgb8 specifically in the cortex in Emx1^{Cre}:Itgb8^{fl/fl} mice (Itgb8-cKO) showed complete absence of homeostatic Tmem119⁺ microglia, while Clec7a was highly expressed in cortical microglia (FIG. 7a). This mouse model allowed us to investigate the effect of Itgb8-deletion in the cortex and hippocampus, while the rest of the brain was not affected. This is specifically important to discriminate microglial responses between affected and unaffected areas within the same brain. Our data validate that MGnD was only induced in the affected areas i.e., cortex and hippocampus. We also observed increased Gfap immunoreactivity colocalized with Clec7a⁺ MGnD microglia in the cortex of Itgb8-cKO mice (FIG. 7b-d). RNAseq of cortical microglia revealed an induction of MGnD-related genes (Apoe, Cd300ld, Cd74 and Axl), while homeostatic genes (Tmem119, Siglech, Mertk and Havcr2) were suppressed (FIG. 7e). Of note, cortical microglia in Itgb8-cKO mice did not express peripheral monocyte lineage gene Ms4a3⁵³, supporting that microglia were not replaced by peripheral monocytes, as shown in the previously published Nestin-CRE:Itgb8^{fl/fl} mouse model⁴⁴. Functional characterization showed induced phagosome formation, antigen presentation, chemokine-signaling, and IFNg signaling in cortical microglia of Itgb8-cKO mice. Additional characterization of microglia using the acute response to neurodegeneration via cranial injection of apoptotic neurons⁶ showed upregulation of antigen-presentation and IFNg signaling related genes in Itgb8-cKO mice as compared to control mice. Furthermore, deletion of Itgb8 promoted microglial Ab phagocytosis in response to acute cranial injection of Ab (FIG. 9A). By focusing on Smad3 as a regulator of microglial homeostatic signature downstream of TGFb^{21, 28, 50}, we found reduced Smad3 phosphorylation and increased Apoe immunoreactivity in Itgb8-KO mice (FIG. 7g-i). Using Cx3cr1-CRE:Smad2/3^{fl/fl} mice, we confirmed that microglial deletion of Smad2/3 resulted in the suppression of microglial homeostatic molecule Tmem119 and enhanced expression of MGnD molecules including Cd68 and Apoe associated with astrocyte activation. The gene expression profile of cortical microglia from Emx1^{Cre}:Itgb8^{fl/fl} mice was highly comparable (R=0.89; P<2.2e-16) to that of Tgfbr2-cKO microglia (FIG. 6j), and previously published microglia transcriptomes from Nrros-KO54 and in Smad2/3-cKO mice (FIG. 10A). Importantly, the AD risk factors including Bin1, Inpp5d, Havcr2 were reduced in all datasets (FIG. 7j, FIG. 10B). The reproducibility in microglial gene expression profiles indicates that regional deletion of Itgb8 suppresses TGFb signaling, leading to an induction of MGnD phenotype.

### Example 8. Microglial Deletion of the Homeostatic Checkpoint Inpp5d Facilitates Plaque Clearance Via the Induction of MGnD Response

The induction of MGnD genes in microglia deleted from Itgb8, Tgfbr2, Nrros or Smad2/3 was associated with the suppression of AD risk factors, including Bin1, Havcr2 and Inpp5d^{3, 4} (FIG. 7j,). In addition, APOE4 microglia showed increased Inpp5d expression levels (FIG. 1a) and enriched Inpp5d locus histone acetylation. To address the impact of microglial specific Inpp5d on AD pathology and MGnD response, we crossed Cx3cr1^{CreERT2/WT}. Inpp5d^{fl/fl} mice to APP/PS1 mice. Microglial deletion of Inpp5d induced MGnD response in APP/PS1 mice was associated with reduced Ab plaque load as determined by Thioflavin-S and HJ3.4b staining. Moreover, microglia deleted with Inpp5d showed increased Clec7a immunoreactivity in association with Ab-plaques. Importantly, Inpp5d deletion in microglia was sufficient to reduce Lamp1⁺ dystrophic neurites in APP/PS1 mice. Furthermore, the increased MGnD response in association with Ab plaques was accompanied by increased Gfap⁺ immunoreactivity following the deletion of microglial Inpp5d. Similarly, recent studies showed that microglial deletion of Inpp5d protects against plaque-induced neuronal dystrophy in transgenic AD mice^{55, 56}. These results support the role of Inpp5d as a microglial homeostatic checkpoint, and that its deletion is beneficial in mitigating AD pathology via the induction of MGnD response.

### Example 9. Blocking ITGB8-TGFb Signaling Enhances MGnD Response and Reduces AD Pathology in APP/PS1 Mice

To test whether enhanced microglia activation and phagocytosis can reduce AD-pathological hallmarks in adult APP/PS1 mice we used anti-ITGB8 neutralizing mAb (ADWA-11)⁵⁷. Three days post injection in 4-month-old APP/PS1 mice, microglia increased antigen-presentation and IFNg signaling (FIG. 7k,l). Immunohistochemical analysis confirmed increased MHC-II⁺ immunoreactivity around the injection site (FIG. 9B). Moreover, 14 d post-injection, we found a significant decrease in Ab plaque size (FIG. 7m,n) in anti-ITGB8 mAb injected group compared to the controls. In APOE4-KI: APP/PS1 mice, treatment with anti-ITGB8 mAb restored the induction of Clec7a and GFAP associated with reduced plaque pathology (FIG. 7o,p). Overall, these results strongly support a critical role of ITGB8-TGFb signaling in the regulation of an MGnD response in AD pathology. Thus, pharmacological targeting of ITGB8-TGFb signaling in AD can promote MGnD response and astrocyte activation and provide a novel approach for therapeutic modulation of innate immunity in AD and dementia.

### Example 10. Targeting ITGB8-TGFb Signaling Mitigates Cognitive Decline in 5×FAD Mice

Microglia and astrocytes play essential roles in brain physiology, but they can also promote central nervous system (CNS) pathology in the context of neurologic diseases. APOE4 is the strongest genetic risk factor for late-onset AD. We established that APOE and TGFβ reciprocally regulate neurodegenerative microglia (MGnD)^{6,22,31}, also known as disease associated microglia (DAM) 7, in pre-clinical models of AD. Astrocytes also respond to neurodegeneration and express a disease-associated astrocyte (DAA) associated with amyloid β (Aβ)-plaques⁴⁰. Moreover, reactive astrocytes were shown to play a beneficial role in limiting AD pathology and depletion of astrocytes in AD mice increased plaque load, synaptic dysfunction, and memory loss⁶⁴. We found that integrin subunit β8 (ITGB8), induced on astrocytes in the APOE4 brain milieu, activates microglial TGFb signaling, locks them in the homeostatic state and thus impairs their response to neurodegeneration. As described in Examples 1-9, we identified the microglia APOE4-ITGB8-TGFb pathway as a negative regulator of microglial response to AD pathology, and restoring MGnD phenotype via blocking ITGB8-TGFb signaling provides a promising therapeutic intervention for AD.

To test whether enhanced microglia activation and phagocytosis can reduce AD-pathological hallmarks in adult APP/PS1 mice, we used an anti-ITGB8 neutralizing mAb (ADWA-11)⁵⁷. Three days post-injection in 4-month-old APP/PS1 mice, microglia increased antigen presentation and IFNg signaling, which we recently showed to play a key role in the induction of pre-MGnD subset restricting neurodegenerative pathology and preserving cognitive function in an AD mouse model¹⁰⁵. Furthermore, 14 days post-injection of anti-ITGB8 mAb, we found a significant decrease in Ab plaque size (see above).

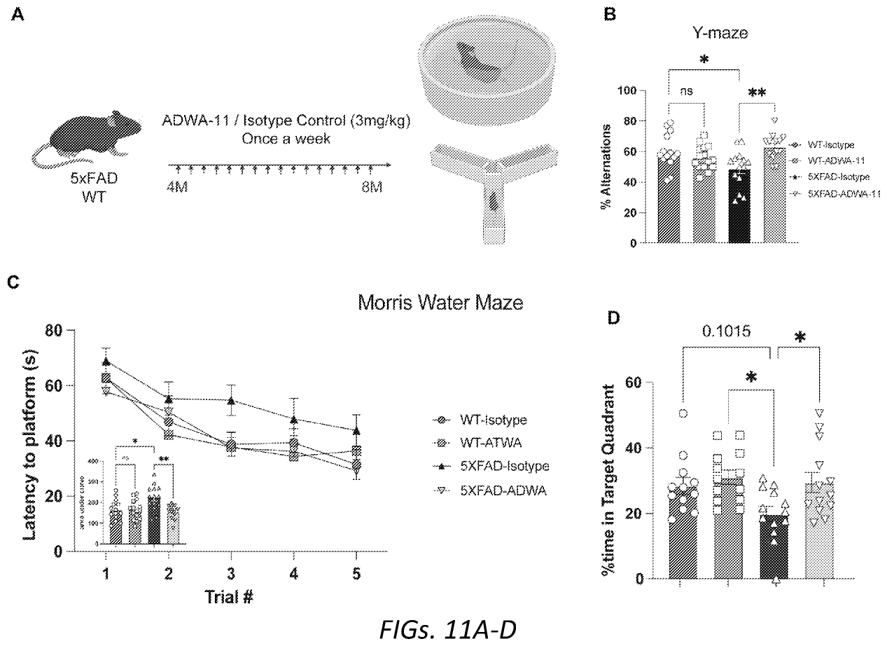

We further addressed whether ADWA-11 can ameliorate the cognitive decline present in AD pathology, treating 5×FAD mice, a widely used model to study amyloid pathology in association with synaptic dysfunction, neurodegeneration, and cognitive impairment¹⁰⁴, along with wild-type mice (n=15/group). The treatment, administered intraperitoneally (ip), commenced in four-month-old mice, once a week using either ADWA-11 mAb or an isotype control (3 mg/ml). Upon reaching eight months of age, we assessed spatial learning and memory via the water maze test, as well as spatial short-term memory and alternation behavior using the T-maze test (FIG. 11A). We found that systemic administration of ADWA-11 restored short-term memory as evidenced by the T-maze test, similar to that of WT mice (FIG. 11B).

Furthermore, spatial learning (FIG. 11C) and memory (FIG. 11D) were also improved following chronic treatment with ADWA-11 mAb. Importantly, the 16-week chronic treatment with ADWA-11 mAb did not yield any visual changes in clinical behavior, providing been safe. Thus, the pharmacological inhibition of ITGB8 signaling by peripheral treatment is a novel approach to treat AD and dementia.

### REFERENCES

1. Colonna, M. & Butovsky, O. Microglia Function in the Central Nervous System During Health and Neurodegeneration. Annu Rev Immunol 35, 441-468 (2017).
2 Heneka, M. T. et al. Neuroinflammation in Alzheimer's disease. Lancet Neurol 14, 388-405 (2015).
3. Jansen, I. E. et al. Genome-wide meta-analysis identifies new loci and functional pathways influencing Alzheimer's disease risk. Nat Genet 51, 404-413 (2019).
4 Wightman, D. P. et al. A genome-wide association study with 1,126,563 individuals identifies new risk loci for Alzheimer's disease. Nat Genet 53, 1276-1282 (2021).
5. Nott, A. et al. Brain cell type-specific enhancer-promoter interactome maps and disease-risk association. Science 366, 1134-1139 (2019).
6. Krasemann, S. et al. The TREM2-APOE Pathway Drives the Transcriptional Phenotype of Dysfunctional Microglia in Neurodegenerative Diseases. Immunity 47, 566-581 e569 (2017).
7. Keren-Shaul, H. et al. A Unique Microglia Type Associated with Restricting Development of Alzheimer's Disease. Cell 169, 1276-1290 e1217 (2017).
8. Farrer, L. A. et al. Effects of age, sex, and ethnicity on the association between apolipoprotein E genotype and Alzheimer disease. A meta-analysis. APOE and Alzheimer Disease Meta Analysis Consortium. JAMA 278, 1349-1356 (1997).
9 Corder, E. H. et al. Gene dose of apolipoprotein E type 4 allele and the risk of Alzheimer's disease in late onset families. Science 261, 921-923 (1993).
10. Strittmatter, W. J. et al. Apolipoprotein E: high-avidity binding to beta-amyloid and increased frequency of type 4 allele in late-onset familial Alzheimer disease. Proc Natl Acad Sci USA 90, 1977-1981 (1993).
11. Raber, J. et al. Isoform-specific effects of human apolipoprotein E on brain function revealed in ApoE knockout mice: increased susceptibility of females. Proc Natl Acad Sci USA 95, 10914-10919 (1998).
12. Youmans, K. L. et al. APOE4-specific changes in Abeta accumulation in a new transgenic mouse model of Alzheimer disease. J Biol Chem 287, 41774-41786 (2012).
13. Liu, D. S. et al. APOE4 enhances age-dependent decline in cognitive function by down-regulating an NMDA receptor pathway in EFAD-Tg mice. Mol Neurodegener 10, 7 (2015).
14. Xu, Q. et al. Profile and regulation of apolipoprotein E (ApoE) expression in the CNS in mice with targeting of green fluorescent protein gene to the ApoE locus. J Neurosci 26, 4985-4994 (2006).
15. Nelissen, K. et al. Liver X receptors regulate cholesterol homeostasis in oligodendrocytes. J Neurosci Res 90, 60-71 (2012).
16. Williams, T., Borchelt, D. R. & Chakrabarty, P. Therapeutic approaches targeting Apolipoprotein E function in Alzheimer's disease. Mol Neurodegener 15, 8 (2020).
17. Basu, S. K., Brown, M. S., Ho, Y. K., Havel, R. J. & Goldstein, J. L. Mouse macrophages synthesize and secrete a protein resembling apolipoprotein E. Proc Natl Acad Sci USA 78, 7545-7549 (1981).
18. Huynh, T. V. et al. Lack of hepatic apoE does not influence early Abeta deposition: observations from a new APOE knock-in model. Mol Neurodegener 14, 37 (2019).
19. Liu, C. C. et al. Peripheral apoE4 enhances Alzheimer's pathology and impairs cognition by compromising cerebrovascular function. Nat Neurosci 25, 1020-1033 (2022).
20. Wang, C. et al. Selective removal of astrocytic APOE4 strongly protects against tau-mediated neurodegeneration and decreases synaptic phagocytosis by microglia. Neuron 109, 1657-1674 e1657 (2021).
21. Butovsky, O. et al. Identification of a unique TGF-beta-dependent molecular and functional signature in microglia. Nat Neurosci 17, 131-143 (2014).
22. Margeta, M. A. et al. Apolipoprotein E4 impairs the response of neurodegenerative retinal microglia and prevents neuronal loss in glaucoma. Immunity 55 (9): 1627-1644.e7 (2022).
23. Fote, G. M. et al. Isoform-dependent lysosomal degradation and internalization of apolipoprotein E requires autophagy proteins. J Cell Sci 135 (2022).
24. Tcw, J. et al. Cholesterol and matrisome pathways dysregulated in astrocytes and microglia. Cell 185, 2213-2233 e2225 (2022).
25. Pohlkamp, T. et al. NHE6 depletion corrects ApoE4-mediated synaptic impairments and reduces amyloid plaque load. Elife 10 (2021).
26. Parkhurst, C. N. et al. Microglia promote learning-dependent synapse formation through brain-derived neurotrophic factor. Cell 155, 1596-1609 (2013).
27. Klein, H. U. et al. Epigenome-wide study uncovers large-scale changes in histone acetylation driven by tau pathology in aging and Alzheimer's human brains. Nat Neurosci 22, 37-46 (2019).
28. Gosselin, D. et al. Environment drives selection and function of enhancers controlling tissue-specific macrophage identities. Cell 159, 1327-1340 (2014).
29. Huang, K. L. et al. A common haplotype lowers PU.1 expression in myeloid cells and delays onset of Alzheimer's disease. Nat Neurosci 20, 1052-1061 (2017).
30. Serrano-Pozo, A., Frosch, M. P., Masliah, E. & Hyman, B. T. Neuropathological alterations in Alzheimer disease. Cold Spring Harb Perspect Med 1, a006189 (2011).
31. Shi, Y. et al. ApoE4 markedly exacerbates tau-mediated neurodegeneration in a mouse model of tauopathy. Nature 549, 523-527 (2017).
32. Yoshiyama, Y. et al. Synapse loss and microglial activation precede tangles in a P301S tauopathy mouse model. Neuron 53, 337-351 (2007).
33. Rosenzweig, N. et al. PD-1/PD-L1 checkpoint blockade harnesses monocyte-derived macrophages to combat cognitive impairment in a tauopathy mouse model. Nat Commun 10, 465 (2019).
34. Radde, R. et al. Abeta42-driven cerebral amyloidosis in transgenic mice reveals early and robust pathology. EMBO Rep 7, 940-946 (2006).
35. Bennett, F. C. et al. A Combination of Ontogeny and CNS

Environment Establishes Microglial Identity. Neuron 98, 1170-1183 e1178 (2018).
36. Van Hove, H. et al. A single-cell atlas of mouse brain macrophages reveals unique transcriptional identities shaped by ontogeny and tissue environment. Nat Neurosci 22, 1021-1035 (2019).
37. Mrdjen, D. et al. High-Dimensional Single-Cell Mapping of Central Nervous System Immune Cells Reveals Distinct Myeloid Subsets in Health, Aging, and Disease. Immunity 48, 380-395 e386 (2018).
38. Jordao, M. J. C. et al. Single-cell profiling identifies myeloid cell subsets with distinct fates during neuroinflammation. Science 363 (2019).
39. Victor, M. B. et al. Lipid accumulation induced by APOE4 impairs microglial surveillance of neuronal-network activity. Cell Stem Cell 29, 1197-1212 e1198 (2022).
40. Habib, N. et al. Disease-associated astrocytes in Alzheimer's disease and aging. Nat Neurosci 23, 701-706 (2020).
41. Nilsson, L. et al. Transthyretin Interferes with Abeta Amyloid Formation by Redirecting Oligomeric Nuclei into Non-Amyloid Aggregates. J Mol Biol 430, 2722-2733 (2018).
42. Argaw, A. T. et al. Astrocyte-derived VEGF-A drives blood-brain barrier disruption in CNS inflammatory disease. J Clin Invest 122, 2454-2468 (2012).
43. Munger, J. S. & Sheppard, D. Cross talk among TGF-beta signaling pathways, integrins, and the extracellular matrix. Cold Spring Harb Perspect Biol 3, a005017 (2011).
44. Arnold, T. D. et al. Impaired alpha Vbeta8 and TGFbeta signaling lead to microglial dysmaturation and neuromotor dysfunction. J Exp Med 216, 900-915 (2019).
45. Gotzl, J. K. et al. Opposite microglial activation stages upon loss of PGRN or TREM2 result in reduced cerebral glucose metabolism. EMBO Mol Med 11 (2019).
46. Wolf, Y., Anderson, A. C. & Kuchroo, V. K. TIM3 comes of age as an inhibitory receptor. Nat Rev Immunol 20, 173-185 (2020).
47. Nakao, A. et al. TGF-beta receptor-mediated signalling through Smad2, Smad3 and Smad4. EMBO J 16, 5353-5362 (1997).
48. Olah, M. et al. Single cell RNA sequencing of human microglia uncovers a subset associated with Alzheimer's disease. Nat Commun 11, 6129 (2020).
49. Zhou, Y. et al. Human and mouse single-nucleus transcriptomics reveal TREM2-dependent and TREM2-independent cellular responses in Alzheimer's disease. Nat Med 26, 131-142 (2020).
50. Gosselin, D. et al. An environment-dependent transcriptional network specifies human microglia identity. Science 356 (2017).
51. Travis, M. A. et al. Loss of integrin alpha (v) beta8 on dendritic cells causes autoimmunity and colitis in mice. Nature 449, 361-365 (2007).
52. Zhang, Y. et al. An RNA-sequencing transcriptome and splicing database of glia, neurons, and vascular cells of the cerebral cortex. J Neurosci 34, 11929-11947 (2014).
53. Liu, Z. et al. Fate Mapping via Ms4a3-Expression History Traces Monocyte-Derived Cells. Cell 178, 1509-1525 e1519 (2019).
54. Wong, K. et al. Mice deficient in NRROS show abnormal microglial development and neurological disorders. Nat Immunol 18, 633-641 (2017).
55. Samuels, J. D. et al. The Alzheimer's disease risk factor INPP5D restricts neuroprotective microglial responses in amyloid beta-mediated pathology. Alzheimers Dement (2023).
56. Iguchi, A. et al. INPP5D modulates TREM2 loss-of-function phenotypes in a beta-amyloidosis mouse model. iScience 26, 106375 (2023).
57. Dodagatta-Marri, E. et al. Integrin alphavbeta8 on T cells suppresses anti-tumor immunity in multiple models and is a promising target for tumor immunotherapy. Cell Rep 36, 109309 (2021).
58. Mahan, T. E. et al. Selective reduction of astrocyte apoE3 and apoE4 strongly reduces Abeta accumulation and plaque-related pathology in a mouse model of amyloidosis. Mol Neurodegener 17, 13 (2022).
59. Rohn, T. T. & Moore, Z. D. Nuclear Localization of Apolipoprotein E4: A New Trick for an Old Protein. Int J Neurol Neurother 4 (2017).
60. Theendakara, V. et al. Direct Transcriptional Effects of Apolipoprotein E. J Neurosci 36, 685-700 (2016).
61. Hollingworth, P. et al. Common variants at ABCA7, MS4A6A/MS4A4E, EPHA1, CD33 and CD2AP are associated with Alzheimer's disease. Nat Genet 43, 429-435 (2011).
62. Pimenova, A. A. et al. Alzheimer's-associated PU.1 expression levels regulate microglial inflammatory response. Neurobiol Dis 148, 105217 (2021).
63. Liddelow, S. A. et al. Neurotoxic reactive astrocytes are induced by activated microglia. Nature 541, 481-487 (2017).
64. Katsouri, L. et al. Ablation of reactive astrocytes exacerbates disease pathology in a model of Alzheimer's disease. Glia 68, 1017-1030 (2020).
65. Tan, Y. et al. Galectin-3: a key player in microglia-mediated neuroinflammation and Alzheimer's disease. Cell Biosci 11, 78 (2021).
66. Sirko, S. et al. Astrocyte reactivity after brain injury-: The role of galectins 1 and 3. Glia 63, 2340-2361 (2015).
67. Boza-Serrano, A. et al. Galectin-3, a novel endogenous TREM2 ligand, detrimentally regulates inflammatory response in Alzheimer's disease. Acta Neuropathol 138, 251-273 (2019).
68. Tao, C. C. et al. Galectin-3 promotes Abeta oligomerization and Abeta toxicity in a mouse model of Alzheimer's disease. Cell Death Differ 27, 192-209 (2020).
69. Mabuchi, F. et al. The apolipoprotein E gene polymorphism is associated with open angle glaucoma in the Japanese population. Mol Vis 11, 609-612 (2005).
70. Lam, C. Y. et al. Association of apolipoprotein E polymorphisms with normal tension glaucoma in a Chinese population. J Glaucoma 15, 218-222 (2006).
71. Margeta, M. A. et al. Association of APOE With Primary Open-Angle Glaucoma Suggests a Protective Effect for APOE epsilon4. Invest Ophthalmol Vis Sci 61, 3 (2020).
72. Tesseur, I. et al. Deficiency in neuronal TGF-beta signaling promotes neurodegeneration and Alzheimer's pathology. J Clin Invest 116, 3060-3069 (2006).
73. Wyss-Coray, T. et al. Amyloidogenic role of cytokine TGF-betal in transgenic mice and in Alzheimer's disease. Nature 389, 603-606 (1997).
74. Kandasamy, M. et al. TGF-beta Signaling: A Therapeutic Target to Reinstate Regenerative Plasticity in Vascular Dementia? Aging Dis 11, 828-850 (2020).
75. Lund, H. et al. Fatal demyelinating disease is induced by monocyte-derived macrophages in the absence of TGF-β signaling. Nat Immunol 19, 1-7 (2018).
76. Kaiser, T. & Feng, G. Tmem119-EGFP and Tmem119-CreERT2 Transgenic Mice for Labeling and Manipulating Microglia. eNeuro 6 (2019).
77. Wang, J. W. et al. Influence of SHIP on the NK repertoire and allogeneic bone marrow transplantation. Science 295, 2094-2097 (2002).
78. Ju, W. et al. Deletion of Smad2 in mouse liver reveals novel functions in hepatocyte growth and differentiation. Mol Cell Biol 26, 654-667 (2006).
79. Li, Q. et al. Redundant roles of SMAD2 and SMAD3 in ovarian granulosa cells in vivo. Mol Cell Biol 28, 7001-7011 (2008).
80. Sullivan, P. M. et al. Targeted replacement of the mouse apolipoprotein E gene with the common human APOE3 allele enhances diet-induced hypercholesterolemia and atherosclerosis. J Biol Chem 272, 17972-17980 (1997).
81. Knouff, C. et al. Apo E structure determines VLDL clearance and atherosclerosis risk in mice. J Clin Invest 103, 1579-1586 (1999).
82. Iwasaki, H. et al. Distinctive and indispensable roles of PU.1 in maintenance of hematopoietic stem cells and their differentiation. Blood 106, 1590-1600 (2005).
83. Proctor, J. M., Zang, K., Wang, D., Wang, R. & Reichardt, L. F. Vascular development of the brain requires beta8 integrin expression in the neuroepithelium. J Neurosci 25, 9940-9948 (2005).
84. Nakawesi, J. et al. alphavbeta8 integrin-expression by BATF3-dependent dendritic cells facilitates early IgA responses to Rotavirus. Mucosal Immunol 14, 53-67 (2021).
85. Gong, S. et al. A gene expression atlas of the central nervous system based on bacterial artificial chromosomes. Nature 425, 917-925 (2003).
86. Gong, S. et al. Targeting Cre recombinase to specific neuron populations with bacterial artificial chromosome constructs. J Neurosci 27, 9817-9823 (2007).
87. Sanmarco, L. M. et al. Gut-licensed IFNgamma (+) NK cells drive LAMP1 (+) TRAIL (+) anti-inflammatory astrocytes. Nature 590, 473-479 (2021).
88. Hao, Y. et al. Integrated analysis of multimodal single-cell data. Cell 184, 3573-3587 e3529 (2021).
89. Ianevski, A., Giri, A. K. & Aittokallio, T. Fully-automated and ultra-fast cell-type identification using specific marker combinations from single-cell transcriptomic data. Nat Commun 13, 1246 (2022).
90. Picelli, S. et al. Smart-seq2 for sensitive full-length transcriptome profiling in single cells. Nat Methods 10, 1096-1098 (2013).
91. Love, M. I., Huber, W. & Anders, S. Moderated estimation of fold change and dispersion for RNA-seq data with DESeq2. Genome Biol 15, 550 (2014).
92. Mootha, V. K. et al. PGC-1alpha-responsive genes involved in oxidative phosphorylation are coordinately downregulated in human diabetes. Nat Genet 34, 267-273 (2003).
93. Subramanian, A. et al. Gene set enrichment analysis: a knowledge-based approach for interpreting genome-wide expression profiles. Proc Natl Acad Sci USA 102, 15545-15550 (2005).
94. Browaeys, R., Saelens, W. & Saeys, Y. NicheNet: modeling intercellular communication by linking ligands to target genes. Nat Methods 17, 159-162 (2020).
95. Gu, Z., Gu, L., Eils, R., Schlesner, M. & Brors, B. circlize Implements and enhances circular visualization in R. Bioinformatics 30, 2811-2812 (2014).
96. Nott, A., Schlachetzki, J. C. M., Fixsen, B. R. & Glass, C. K. Nuclei isolation of multiple brain cell types for omics interrogation. Nat Protoc 16, 1629-1646 (2021).
97. Langmead, B. & Salzberg, S. L. Fast gapped-read alignment with Bowtie 2. Nat Methods 9, 357-359 (2012).
98. Tarasov, A., Vilella, A. J., Cuppen, E., Nijman, I. J. & Prins, P. Sambamba: fast processing of NGS alignment formats. Bioinformatics 31, 2032-2034 (2015).
99. Li, H. et al. The Sequence Alignment/Map format and SAMtools. Bioinformatics 25, 2078-2079 (2009).
100. Ramirez, F., Dundar, F., Diehl, S., Gruning, B. A. & Manke, T. deepTools: a flexible platform for exploring deep-sequencing data. Nucleic Acids Res 42, W187-191 (2014).
101. Zhang, Y. et al. Model-based analysis of ChIP-Seq (MACS). Genome Biol 9, R137 (2008).
102. Bero, A. W. et al. Bidirectional relationship between functional connectivity and amyloid-beta deposition in mouse brain. J Neurosci 32, 4334-4340 (2012).
103. Li, Q. et al. Developmental Heterogeneity of Microglia and Brain Myeloid Cells Revealed by Deep Single-Cell RNA Sequencing. Neuron 101, 207-223 e210 (2019).
104. Oakley, H. et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140, doi: 10.1523/JNEUROSCI.1202-06.2006 (2006).
105. Yin, Z. et al., Identification of a protective microglial state mediated by miR-155 and interferon-γ signaling in a mouse model of Alzheimer's disease. Nat Neurosci. 2023 July; 26 (7): 1196-1207.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a neurodegenerative condition associated with microglial impairment, the method comprising administering a therapeutically effective amount of an inhibitor of Integrin Subunit beta 8 (ITGB8).

2. The method of claim 1, wherein the inhibitor of ITGB8 is an antibody that binds to ITGB8.

3. The method of claim 2, wherein the antibody that binds to ITGB8 is ADWA11, ADWA16, C6D4, 37E11, HuC6D4F12, CL7290 or a humanized version thereof.

4. The method of claim 3, wherein the humanized version is ADWA11-2.1, ADWA11-2.2, ADWA11-2.3, ADWA11-2.4, ADWA16-1, ADWA16-2, ADWA16-3, ADWA16-3.2, ADWA16-4, ADWA16hugraft, Ab1, Ab2, or Ab3.

5. The method of claim 1, wherein the inhibitor of ITGB8 is an inhibitory oligonucleotide targeting human ITGB8 that decreases ITGB8 expression.

6. The method of claim 5, wherein the oligonucleotide is 15 to 21 nucleotides in length.

7. The method of claim 5, wherein at least one nucleotide of the oligonucleotide is a nucleotide analogue.

8. The method of claim 5, wherein the oligonucleotide is a gapmer or a mixmer.

9. The method of claim 1, wherein the neurodegenerative condition is Alzheimer's disease (AD) or amyotrophic lateral sclerosis (ALS).

10.-18. (canceled)
